SEQUENCE LISTING (2 sequences)

SEQ ID NO: 1 - DNA, 62 bp, synthetic construct
  source 1..62: mol_type = other DNA
tagctagcatgtacccatacgacgtcccagactacgctatggaggtgacggcggaccagccg

SEQ ID NO: 2 - DNA, 33 bp, synthetic construct
  source 1..33: mol_type = other DNA
tgcggccgcctaacccatggcggtgaccatgct

(19) United States
(12) Patent Application Publication
(10) Pub. No.: US 2026/0191831 A1
(43) Pub. Date: Jul. 9, 2026

(54) GATA TRANSCRIPTION FACTOR PROTEOLYSIS TARGETING CHIMERA COMPOUND AND PREPARATION METHOD AND USE THEREOF

(71) Applicant: Sida SHIDAI Pharmaceutical (Suzhou) Co., Ltd., Suzhou (CN)

(72) Inventors: Liming CHEN, Nanjing (CN); Yu HUANG, Nanjing (CN)

(73) Assignee: Sida SHIDAI Pharmaceutical (Suzhou) Co., Ltd., Suzhou (CN)

(21) Appl. No.: 19/011,242

(22) Filed: Jan. 6, 2025

(51) Int. Cl. A61K 31/427; A61P 35/00; C07D 417/14

(52) U.S. Cl. CPC A61K 31/427; A61P 35/00; C07D 417/14

(57) ABSTRACT

The present disclosure discloses a GATA transcription factor proteolysis targeting chimera compound, a preparation method and a use thereof. The compound has a structure represented by formula I. The compound has a binding capacity with a GATA transcription factor at nanomolar level, a significant degradation effect on the GATA transcription factor, and a remarkable inhibitory effect on the tumor growth, which can be effectively used in the preparation of a drug for degrading the GATA transcription factor, a drug for treating the GATA transcription factor abnormalities, and a drug for treating cancer.

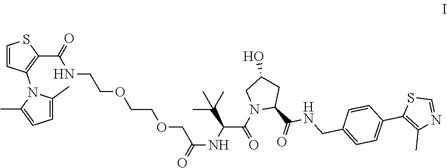 [SMILES: Cc1ncsc1-c1ccc(CNC(=O)[C@@H]2C[C@@H](O)CN2C(=O)[C@@H](NC(=O)COCCOCCNC(=O)c2sccc2-n2c(C)ccc2C)C(C)(C)C)cc1]

### REFERENCE TO SEQUENCE LISTING

The contents of the electronic sequence listing (SDYY-20240157-I-US.xml; Size: 2,772 bytes; and Date of Creation: May 14, 2025) is herein incorporated by reference in its entirety.

### TECHNICAL FIELD

The present disclosure belongs to the field of biomedicine, specially to a GATA transcription factor proteolysis targeting chimera compound, a preparation method and a use thereof.

### BACKGROUND

Human GATA transcription factors include several members (six classic GATA transcription factors of GATA1 to 6 and a non-classical GATA transcription factor of TRPS1), all of which play important biological role in the development of many diseases by their GATA zinc finger structure recognizing the GATA elements on DNA to regulate downstream gene expression. GATA transcription factors promote the malignant progression of various cancers and are important carcinogens. Take GATA3, a typical GATA family member, as an example. GATA3 is often highly expressed in patients with breast cancer, which not only serves as a pioneer transcription factor to drive the expression of ER-related genes by recruiting other cofactors, but also can promote the G1/S transition by upregulating genes such as CCND1 to promote the proliferation of breast cancer cells. At the same time, researchers found that the dual deletion of GATA3 and MDM2 can lead to synthetic lethality of ER-positive breast cancer cells. In the cervical squamous cell carcinomas, GATA3 can stabilize the invasiveness of HIT-1α-enhanced cancers. In the T cell tumors, GATA3 can also serve as a proto-oncogene to promote the growth and survival of tumor cells. Therefore, GATA transcription factors such as GATA3 are potential therapeutic targets. In addition, GATA transcription factor abnormalities are also important cause of anemia, hypoparathyroidism, deafness and infertility, as well as development-related diseases caused by kidney and heart defects. However, all the transcription factors, including GATA transcription factors, lack structurally stable small molecule binding pockets and allosteric regulatory sites, exhibit positive charge enrichment and convex shape on their transcription factor characteristic DNA binding domain surfaces, and have been considered as “undruggable” protein. Proteolysis targeting chimera, PROTAC, technology achieves a specific degradation by means of utilizing an in vivo proteasome system (UPS) to spatially draw the target protein closer to a specific E3 ubiquitinated ligase, so that it is possible to target an undruggable protein. Currently, a variety of PROTAC drugs have entered clinical practice. However, there is no GATA transcription factor proteolysis targeting chimera drug yet.

### SUMMARY

Purpose of the present disclosure: To solve the existing problems in the prior art, the present disclosure provides a proteolysis targeting chimera compound targeting a GATA transcription factor. It is the first time to propose a compound drug targeting the degradation of the GATA transcription factor, which has high affinity with the target protein and high efficacy, and can be used to prepare a drug for treating breast cancer.

The present disclosure further provides a preparation method and a use of the compound. The present disclosure provides a new synthetic route of VHL ligands, which successfully connects three core components of the drug together, and further breaks through the technical bottleneck of undruggability of the transcription factors in the existing technology.

Technical solutions: In order to achieve the above purpose, the present disclosure provides a GATA transcription factor proteolysis targeting chimera compound and pharmaceutically acceptable salts thereof, where the compound has a structure represented by formula (I):

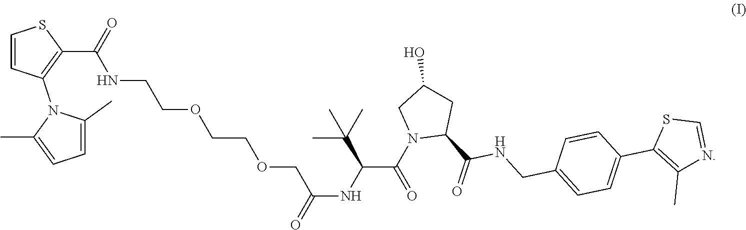 [SMILES: Cc1ncsc1-c1ccc(CNC(=O)[C@@H]2C[C@@H](O)CN2C(=O)[C@@H](NC(=O)COCCOCCNC(=O)c2sccc2-n2c(C)ccc2C)C(C)(C)C)cc1]

The present disclosure provides a method for preparing the GATA transcription factor proteolysis targeting chimera compound and pharmaceutically acceptable salts thereof, which having a reaction process of:

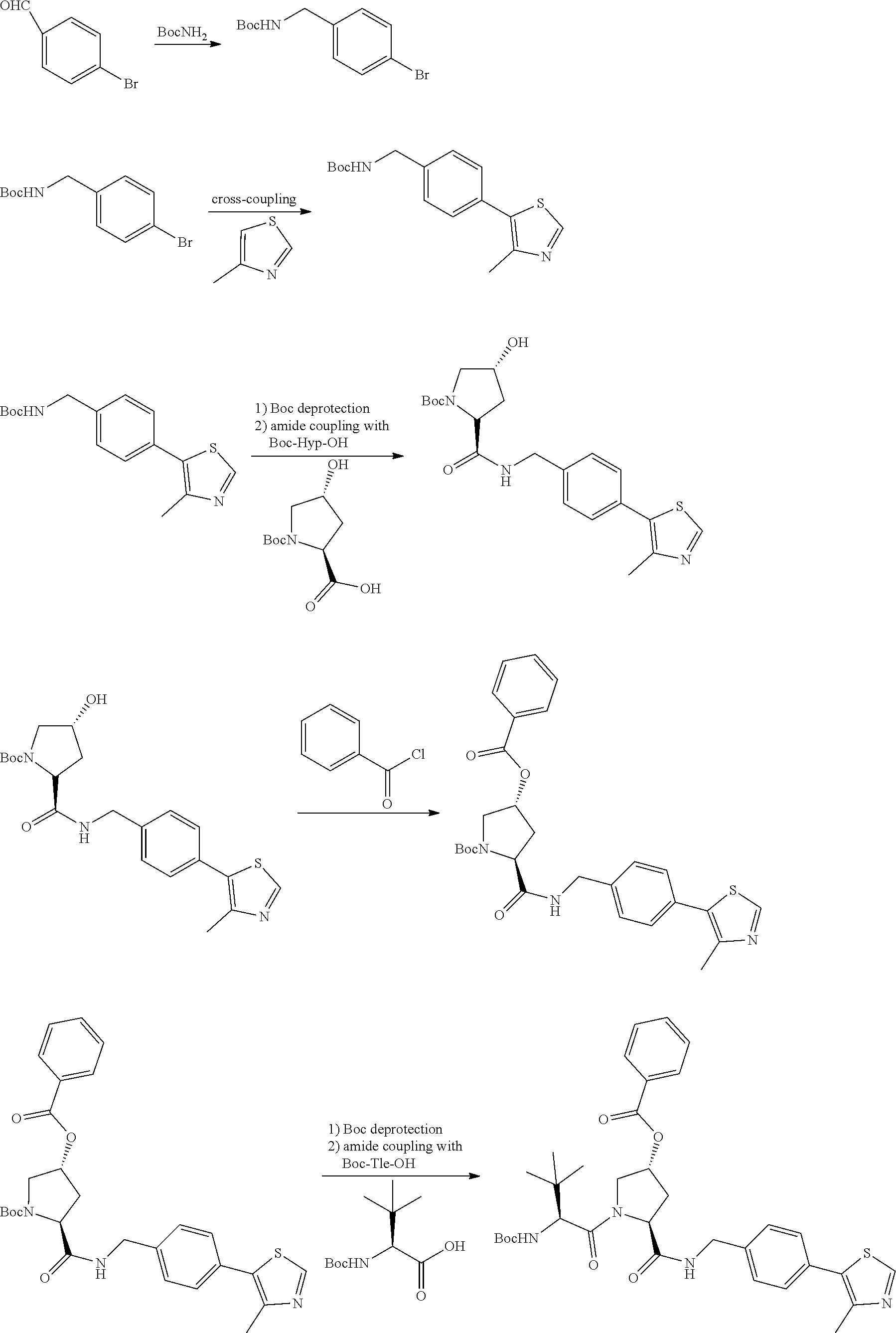

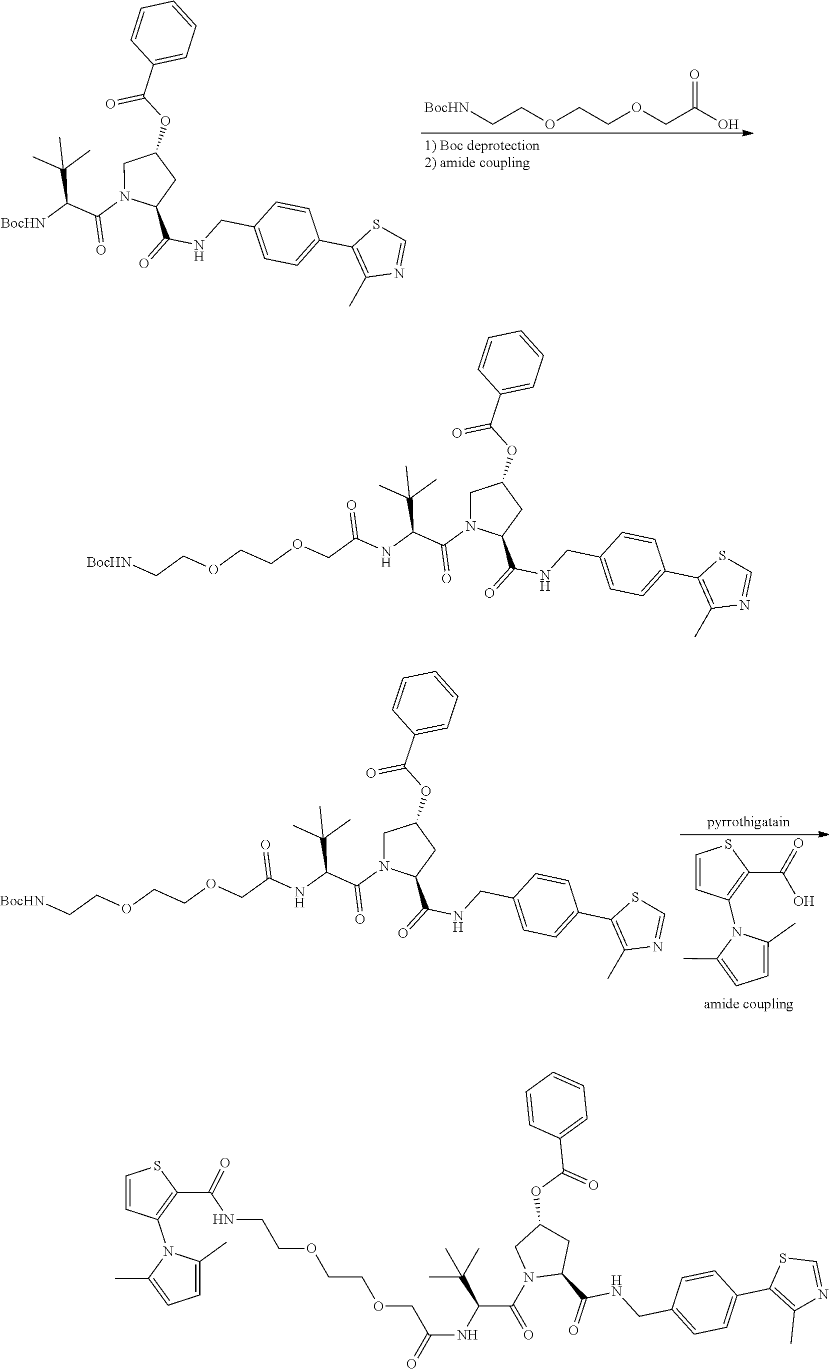

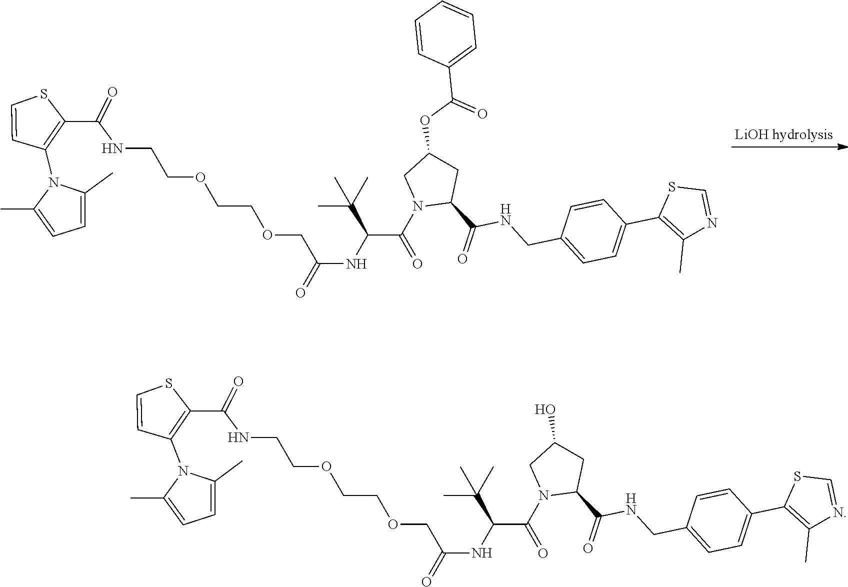 [SMILES: Cc1ncsc1-c1ccc(CNC(=O)[C@@H]2C[C@@H](OC(=O)c3ccccc3)CN2C(=O)[C@@H](NC(=O)COCCOCCNC(=O)c2sccc2-n2c(C)ccc2C)C(C)(C)C)cc1 ; Cc1ncsc1-c1ccc(CNC(=O)[C@@H]2C[C@@H](O)CN2C(=O)[C@@H](NC(=O)COCCOCCNC(=O)c2sccc2-n2c(C)ccc2C)C(C)(C)C)cc1]

Where, the synthetic method includes the following steps:
(1) slowly and sequentially dropwise adding Et₃SiH and TFA to tert-butyl carbamate and 4-bromobenzaldehyde in a solvent, stirring to react, then quenching the reaction, then extracting, washing, drying, filtering, and concentrating in vacuum to obtain a crude product, and isolating and purifying the crude product to obtain an intermediate product 1;
(2) dissolving the intermediate product 1, 4-methylthiazole, Pd(OAc)₂, and K₂CO₃ in N,N-dimethylacetamide, heating to react, then cooling to room temperature, diluting, then extracting, washing, drying, filtering, and concentrating in vacuum to obtain a crude product, and isolating and purifying the crude product to obtain an intermediate product 2;
(3) dissolving the intermediate product 2 (1 mmol) in DCM, adding TFA, stirring to react, then extracting, washing, drying to obtain a crude deprotected amine, and dissolving the crude deprotected amine in N,N-dimethylformamide, adding boc-L-hydroxyproline, then adding DIPEA and HATU sequentially while stirring to react, then adding water, extracting, washing, drying, filtering, and concentrating in vacuum to obtain a crude product, and isolating and purifying the crude product to obtain an intermediate product 3;
(4) dissolving the intermediate product 3, benzoyl chloride and DMAP in DCM, and adding TEA under an ice bath condition, then stirring to react, adding H₂O, extracting, washing, drying, filtering, and concentrating in vacuum to obtain a crude product, and isolating and purifying the crude product to obtain an intermediate product 4;
(5) dissolving the intermediate product 4 in DCM, adding TFA, stirring to react, extracting, washing, and drying to obtain a crude deprotected amine, dissolving the crude deprotected amine in N,N-dimethylformamide, adding boc-L-tert-leucine, then adding DIPEA and HATU sequentially while stirring to react, then adding water, extracting, washing, drying, filtering, and concentrating in vacuum to obtain a crude product, and isolating and purifying the crude product to obtain an intermediate product 5;
(6) dissolving the intermediate product 5 in DCM, adding TFA, stirring to react, extracting, washing, and drying to obtain a crude deprotected amine, dissolving the crude deprotected amine in N,N-dimethylformamide, adding 2-[2-(tert-butoxycarbonylamino) ethoxy]ethoxy acetic acid, then adding DIPEA and HATU sequentially while stirring to react, then adding water, extracting, washing, drying, filtering, and concentrating in vacuum to obtain a crude product, and isolating and purifying the crude product to obtain a reactant 1;
(7) dissolving the reactant 1 in DCM, adding TFA, stirring to react, extracting, washing, and drying to obtain a crude deprotected amine, dissolving the crude deprotected amine in N,N-dimethylformamide, adding methyl 3-(2,5-dimethyl-1H-pyrrol-1-yl)-2-thiophene carboxylate, then adding DIPEA and HATU sequentially while stirring to react, then adding water, extracting, washing, drying, filtering, and concentrating in vacuum to obtain a crude product, and isolating and purifying the crude product to obtain an intermediate product 6; and
(8) dissolving the intermediate product 6 in THE, placing a prepared aqueous solution of lithium hydroxide into the mixture under an ice bath condition, stirring to react, after neutralizing the mixture, extracting, drying, filtering, and concentrating in vacuum to obtain a crude product, and isolating and purifying the crude product to obtain the GATA transcription factor proteolysis targeting chimera compound.

The present disclosure provides a use of the compound and pharmaceutically acceptable salts thereof in the preparation of a drug for degrading a GATA transcription factor or a drug for treating GATA transcription factor abnormalities.

Where, the use of the compound and pharmaceutically acceptable salts thereof in the preparation of a degradation agent for degrading a GATA3 protein.

The present disclosure provides a use of the compound and pharmaceutically acceptable salts thereof in the preparation of a drug for treating cancer.

Where, the use of the compound and pharmaceutically acceptable salts thereof in the preparation of a drug for treating breast cancer.

The present disclosure provides a method for degrading a GATA transcription factor or treating GATA transcription factor abnormalities in a subject in need thereof, which comprising administering to the subject a therapeutically-effective amount of the compound.

The present disclosure provides a method for degrading a GATA3 protein in a subject in need thereof, which comprising administering to the subject a therapeutically-effective amount of the compound.

The present disclosure provides a method for treating cancer in a subject in need thereof, which comprising administering to the subject a therapeutically-effective amount of the compound.

Where, the cancer is breast cancer.

The present disclosure provides a pharmaceutical composition of the GATA transcription factor proteolysis targeting chimera compound, comprising the compound or pharmaceutically acceptable salts thereof, and a pharmaceutically acceptable carrier.

Where, the pharmaceutical composition is capsule, powder, tablet, granule, pill, injection, syrup, oral liquid, inhalant, ointment, suppository or patch.

The present disclosure provides a method for degrading a GATA transcription factor or treating GATA transcription factor abnormalities in a subject in need thereof, which comprising administering to the subject a therapeutically-effective amount of the composition.

The present disclosure provides a method for degrading a GATA3 protein in a subject in need thereof, which comprising administering to the subject a therapeutically-effective amount of the composition.

The present disclosure provides a method for treating cancer in a subject in need thereof, which comprising administering to the subject a therapeutically-effective amount of the composition.

Where, the cancer is breast cancer.

The GATA transcription factor proteolysis targeting chimera compound (PROTAC) of the present disclosure has a binding capacity with a GATA transcription factor at nanomolar level, a significant degradation effect on the GATA transcription factor, and a remarkable inhibitory effect on the tumor growth, which can be effectively used in the preparation of a drug for degrading the GATA transcription factor, a drug for treating the GATA transcription factor abnormalities, and a drug for treating cancer.

By specific design and screening, the present disclosure first achieves the synthesis of compounds targeting the degradation of GATA, and effectively breaks through the technical bottleneck that the transcription factors are considered as undruggable drug targets in the prior art. The present disclosure designs a new synthetic route for existing VHL ligands and newly discovered GATA ligands and the synthetic splicing of three components of the final drug; and synthesizes the first drug targeting the degradation of GATA transcription factors which are then used to treat breast cancer with remarkable effect.

The compound of the present disclosure contains three core component compounds having structures represented by formula (II), formula (III), and formula (IV), respectively:
GATA protein binding compound (II):

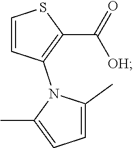

Linker (III):

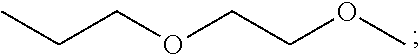

VHL ligand (IV):

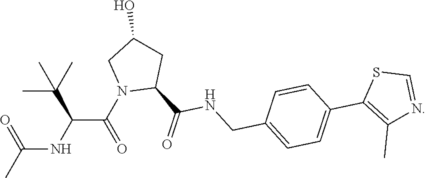 [SMILES: CC(=O)N[C@H](C(=O)N1C[C@H](O)C[C@H]1C(=O)NCc1ccc(-c2scnc2C)cc1)C(C)(C)C]

Beneficial effects: Compared with the prior art, the present disclosure has the following advantages:
1. The present disclosure first proposes and synthesizes a compound drug targeting the degradation of GATA transcription factors, which has a very strong binding force with GATA3, can be used in the preparation of a drug for degrading GATA transcription factors or a drug for treating GATA transcription factor abnormalities, and can further be used to treat diseases such as tumors caused by GATA transcription factor abnormalities with very good effects.
2. The present disclosure proposes a new synthetic route for VHL ligands, and provides a new synthetic route for splicing the three components of a transcription factor proteolysis targeting chimera compound.
3. Compared with the prior art of intervening with the gene expression of the GATA transcription factors by gene knockout and RNA interference to achieve the down-regulation of the GATA protein level, the present disclosure provides a compound which can directly reduce the GATA protein level, as well as a brand-new technology.
4. Compared with the GATA ligand compound (formula II) alone, the GATA transcription factor proteolysis targeting chimera compound synthesized in the present disclosure has a target protein affinity increased by more than 10,000 times and a drug efficacy increased by 40 to 70 times; while the compound without GATA ligand has no activity.
5. The compound of the present disclosure is ingenious in design, simple in structure, cheap and easily available in raw materials, safe and environmentally friendly in synthesis process, and easy for large-scale production.

### BRIEF DESCRIPTION OF THE DRAWINGS

FIG. 1 is an NMR spectrum of an intermediate product 1;

FIG. 2 is an NMR spectrum of an intermediate product 2;

FIG. 3 is an NMR spectrum of an intermediate product 3;

FIG. 4 is an NMR spectrum of an intermediate product 4;

FIG. 5 is an NMR spectrum of an intermediate product 5;

FIG. 6 is a high-resolution image of reactant 1;

FIG. 7 is a high-resolution image of an intermediate product 6;

FIG. 8 is an NMR spectrum of the GATA transcription factor proteolysis targeting chimera compound of formula (I);

FIG. 9 is a diagram showing the MST experimental results of binding force of the GATA transcription factor proteolysis targeting chimera compound of formula (I) and the individual GATA ligand Pyrrothigatain compound of formula (II) to GATA3, where the binding force is as shown;

FIG. 10 shows the expression profile of the GATA3 in each type of breast cancer;

FIG. 11 is a diagram showing the degradation of the GATA transcription factor proteolysis targeting chimera compound on the GATA3 protein in T47D and MCF7 cells;

FIG. 12 is a diagram of the GATA3 protein recovery in T47D and MCF7 cells;

FIG. 13 is a concentration diagram of the GATA transcription factor proteolysis targeting chimera compound resulting in 50% lethal rate of T47D;

FIG. 14 is a concentration diagram of the GATA transcription factor proteolysis targeting chimera compound resulting in 50% lethal rate of MCF7;

FIG. 15 is a diagram showing the ability of the GATA transcription factor proteolysis targeting chimera compound inhibiting the proliferation of T47D cells;

FIG. 16 is a diagram showing the ability of the GATA transcription factor proteolysis targeting chimera compound inhibiting the proliferation of MCF7 cells;

FIG. 17 is a diagram showing the flow cytometry results of the GATA transcription factor proteolysis targeting chimera compound promoting the apoptosis of T47D cells;

FIG. 18 is a diagram showing the flow cytometry results of the GATA transcription factor proteolysis targeting chimera compound promoting the apoptosis of MCF7 cells;

FIG. 19 is a diagram showing the protein results of the GATA transcription factor proteolysis targeting chimera compound promoting the apoptosis of T47D and MCF7 cells;

FIG. 20 is a diagram showing the results of normal saline and GATA transcription factor proteolysis targeting chimera compound inhibiting the breast tumor growth in mice;

FIG. 21 is a diagram showing the change in weight of mice treated with normal saline and GATA transcription factor proteolysis targeting chimera compound; and

FIG. 22 is a diagram showing the volume and weight of tumors stripped from mice treated with normal saline and GATA transcription factor proteolysis targeting chimera compound.

In the above drawings, the GATA transcription factor proteolysis targeting chimera compound is represented by Degrader or the drug concentration is directly shown.

### DESCRIPTION OF THE EMBODIMENTS

The present disclosure will be further illustrated below by reference to specific embodiments.

The experimental methods described in the embodiments are all conventional methods unless otherwise specified; and the reagents and materials are all commercially available unless otherwise specified.

### Embodiment 1

### Preparation of the GATA Transcription Factor Proteolysis Targeting Chimera Compound

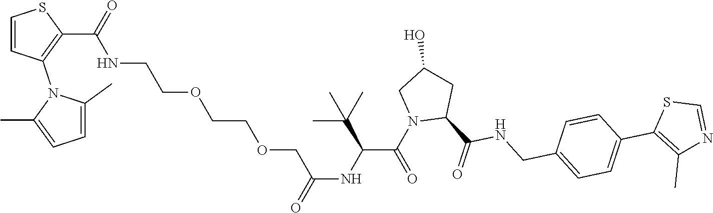 [SMILES: Cc1ncsc1-c1ccc(CNC(=O)[C@@H]2C[C@@H](O)CN2C(=O)[C@@H](NC(=O)COCCOCCNC(=O)c2sccc2-n2c(C)ccc2C)C(C)(C)C)cc1]

### 1. Preparation of Reactant 1

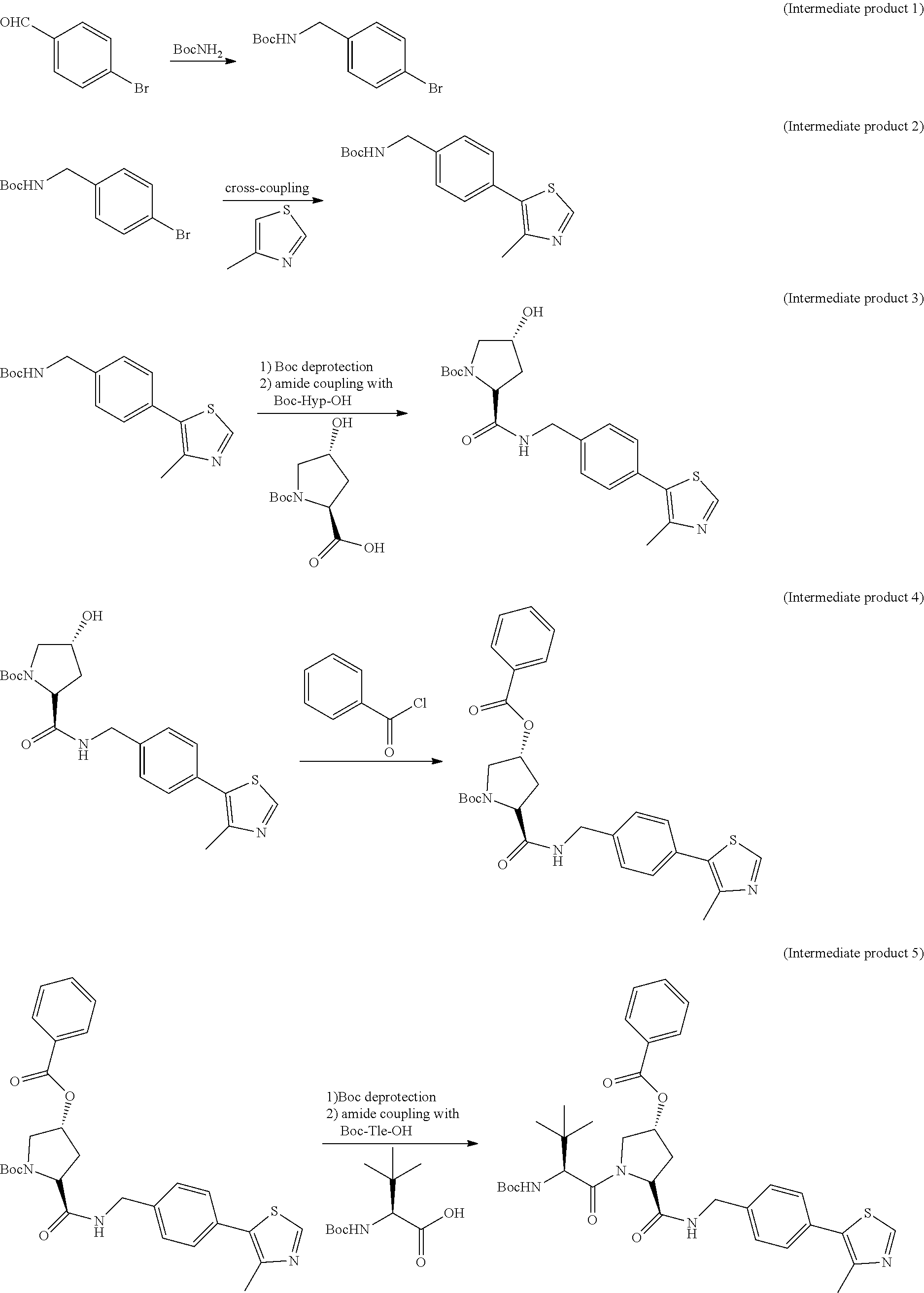

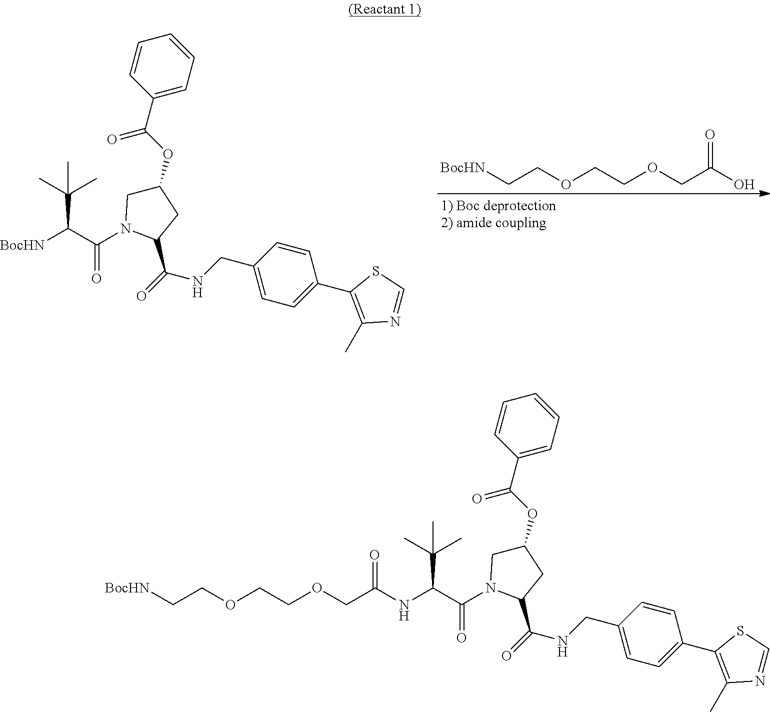

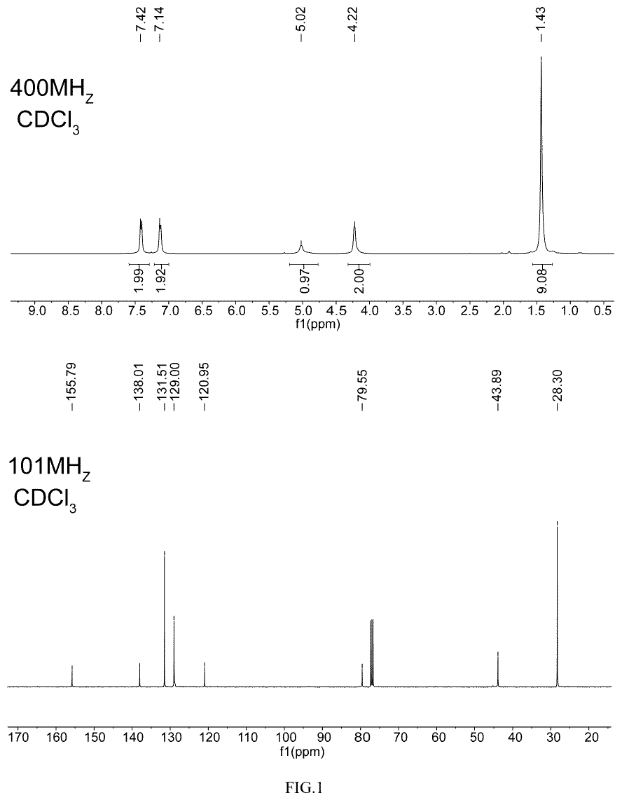

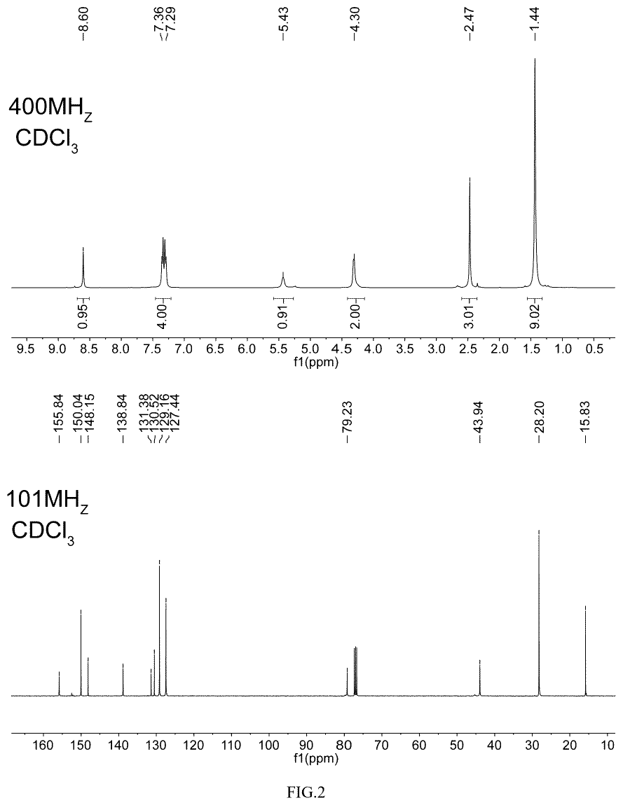

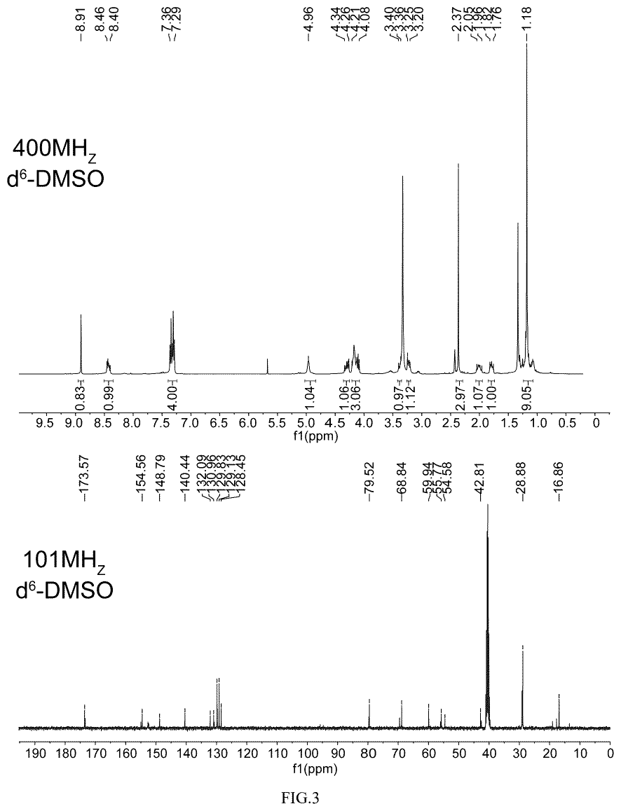

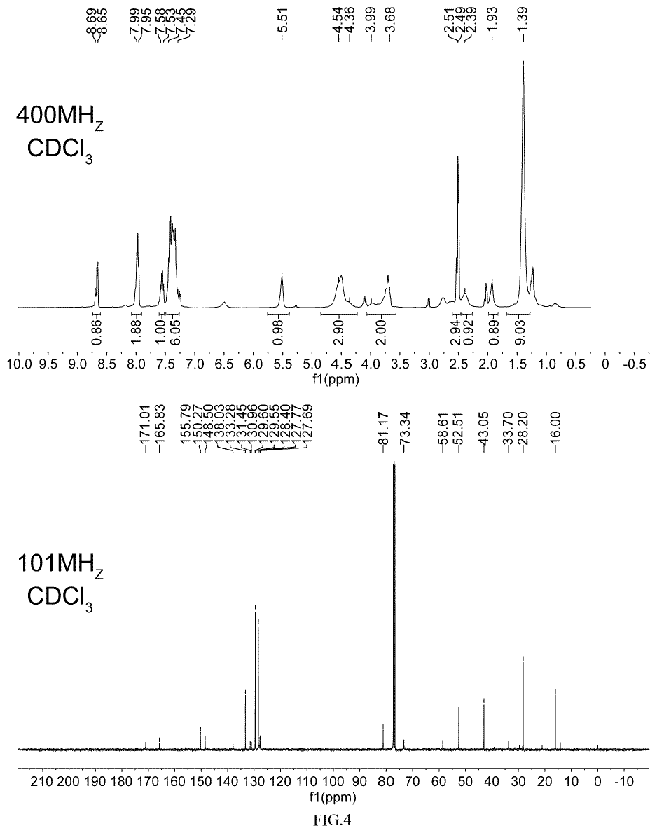

(1) Under nitrogen protection, Et₃SiH (3 mmol) and TFA (2 mmol) were slowly dropwise added to tert-butyl carbamate (3 mmol) and 4-bromobenzaldehyde (1 mmol) in solvent of DCM (2 mL) and MeCN (6 mL). The mixture was stirred at room temperature for 48 hours. Then, saturated NaHCO₃ solution (2 mL) was added to the mixture to quench the reaction, and the reaction was extracted with DCM (3×5 mL). Sequentially, the reaction was washed with saturated brine (2 mL), and the organic phase was dried over Na₂SO₄, filtered, and concentrated in vacuum to obtain a crude product. The crude product was purified by column chromatography on silica gel (petroleum ether) to obtain an intermediate product 1. The NMR results are shown in FIG. 1. ¹H NMR (400 MHz, CDCl₃) δ 7.42 (d, J=7.6 Hz, 2H), 7.14 (d, J=7.2 Hz, 2H), 5.02 (brs, 1H), 4.22 (s, 2H), 1.43 (s, 9H). ¹³C NMR (101 MHz, CDCl₃) δ 155.8, 138.0, 131.5, 129.0, 121.0, 79.6, 43.9, 28.3.
(2) Under nitrogen protection, the intermediate product 1 (1 mmol), 4-methylthiazole (2 mmol), Pd(OAc)₂ (0.01 mmol), and K₂CO₃ (2 mmol) were dissolved in N,N-dimethylacetamide (1 mL), heated to 130° C. under nitrogen atmosphere for a reaction time of 4 hours. Then, the reaction was cooled to room temperature, diluted with water (4 mL), and extracted with DCM (3×5 mL). Then, the organic phase was sequentially washed with saturated brine (2 mL), dried over Na₂SO₄, filtered, and concentrated in vacuum to obtain a crude product. The crude product was purified by column chromatography on silica gel (petroleum ether) to obtain an intermediate product 2. The NMR results are shown in FIG. 2. ¹H NMR (400 MHz, CDCl₃) δ 8.60 (s, 1H), 7.36-7.29 (m, 4H), 5.43 (brs, 1H), 4.30 (s, 2H), 2.47 (s, 3H), 1.44 (s, 9H). ¹³C NMR (101 MHz, CDCl₃) δ 155.8, 150.0, 148.2, 138.8, 131.4, 130.5, 129.2, 127.4, 79.2, 43.9, 28.2, 15.8.
(3) Under nitrogen protection, the intermediate product 2 (1 mmol) was dissolved in DCM (5 mL), and TFA (5 mL) was added. The reaction was stirred at room temperature for 2 hours, and saturated NaHCO₃ solution was added to completely remove TFA. The organic phase was extracted with DCM (3×5 mL), and then sequentially washed with saturated brine (2 mL), dried over Na₂SO₄, and the oily residue was further dried in high vacuum to obtain a total product of crude deprotected amine, which was dissolved in N,N-dimethylformamide (5 mL), boc-L-hydroxyproline (1 mmol) was added, and DIPEA (4 mmol) was added under stirring. 5 min later, HATU (1.1 mmol) was added. The reaction was stirred at room temperature for 18 hours. Water (50 mL) was added, and the reaction was extracted with EtOAc (3×25 mL). Then, the organic phase was sequentially washed with saturated brine (10 mL), dried over Na₂SO₄, filtered, and concentrated in vacuum to obtain a crude product. The crude product was purified by column chromatography on silica gel (petroleum ether) to obtain an intermediate product 3. The NMR results are shown in FIG. 3. ¹H NMR (400 MHz, d⁶-DMSO) δ 8.91 (s, 1H), 8.46-8.40 (m, 1H), 7.36-7.29 (m, 4H), 4.96 (brs, 1H), 4.34-4.26 (m, 1H), 4.21-4.08 (m, 3H), 3.40-3.36 (m, 1H), 3.25-3.20 (m, 1H), 2.37 (s, 3H), 2.05-1.96 (m, 1H), 1.82-1.76 (m, 1H), 1.18 (s, 9H). ¹³C NMR (101 MHz, d₆-DMSO) δ 173.6, 154.6, 148.8, 140.4, 132.1, 131.0, 129.8, 129.1, 128.5, 79.5, 68.8, 59.9, 55.8, 54.6, 42.8, 28.9, 16.9.
(4) Under nitrogen protection, the intermediate product 3 (1 mmol), benzoyl chloride (1.5 mmol), and DMAP (1.1 mmol) were dissolved in DCM (15.3 mL). At 0° C., TEA (5 mmol) was added. The reaction was stirred for 30 min, warmed to room temperature, and stirred for additional 5 hours. H₂O (4 mL) was added, and the organic phase was extracted with DCM (3×5 mL). Then, the organic layer was sequentially washed with saturated brine (2 mL), dried over Na₂SO₄, filtered, and concentrated in vacuum to obtain a crude product. The crude product was purified by column chromatography on silica gel (petroleum ether) to obtain an intermediate product 4. The NMR results are shown in FIG. 4. ¹H NMR (400 MHz, CDCl₃) δ 8.69-8.65 (m, 1H), 7.99-7.95 (m, 2H), 7.58-7.53 (m, 1H), 7.45-7.29 (m, 6H), 5.51 (s, 1H), 4.54-4.36 (m, 3H), 3.99-3.68 (m, 2H), 2.51-2.49 (m, 3H), 2.39 (brs, 1H), 1.93 (brs, 1H), 1.39 (s, 9H). ¹³C NMR (101 MHz, CDCl₃) δ 171.0, 165.8, 155.8, 150.3, 148.5, 138.0, 133.3, 131.5, 131.0, 129.6 (2C), 128.4, 127.8, 127.7, 81.2, 73.3, 58.6, 52.5, 43.1, 33.7, 28.2, 16.0.
(5) Under nitrogen protection, the intermediate product 4 (1 mmol) was dissolved in DCM (5 mL), and TFA (5 mL) was added. The reaction was stirred at room temperature for 2 hours, and saturated NaHCO₃ solution was added to completely remove TFA. The organic phase was extracted with DCM (3×5 mL), and then sequentially washed with saturated brine (2 mL), dried with Na₂SO₄, and the oily residue was further dried in high vacuum to obtain a total product of crude deprotected amine, which was dissolved in N,N-dimethylformamide (5 mL), boc-L-tert-leucine (1 mmol) was added, and DIPEA (4 mmol) was added under stirring. 5 min later, HATU (1.1 mmol) was added. The reaction was stirred at room temperature for 18 hours. Water (50 mL) was added, and the reaction was extracted with EtOAc (3×25 mL). Then, the organic phase was sequentially washed with saturated brine (10 ml), dried with Na₂SO₄, filtered and concentrated in vacuum to obtain a crude product. The crude product was purified by column chromatography on silica gel (petroleum ether) to obtain an intermediate product 5. The NMR results are shown in FIG. 5. ¹H NMR (400 MHz, CDCl₃) δ 8.68 (s, 1H), 8.00 (d, J=7.6 Hz, 1H), 7.53 (t, J=7.2 Hz, 1H), 7.41-7.34 (m, 7H), 7.29 (t, J=5.2 Hz, 1H), 5.59 (s, 1H), 5.12 (d, J=9.6 Hz, 1H), 4.80 (t, J=7.6 Hz, 1H), 4.59 (dd, J=14.8, 6.8 Hz, 1H), 4.37-4.20 (m, 3H), 3.86 (dd, J=11.6, 4 Hz, 1H), 2.92-2.85 (m, 1H), 2.51 (s, 3H), 2.40-2.35 (m, 1H). 1.28 (s, 9H), 0.90 (s, 9H). ¹³C NMR (101 MHz, CDCl₃) δ 172.6, 170.0, 166.0, 155.6, 150.3, 148.4, 137.8, 133.2, 131.6, 131.0, 129.7, 129.6, 128.3, 128.2, 79.6, 73.5, 58.8, 58.3, 53.8, 43.4, 35.1, 33.0, 28.1, 26.3, 25.3, 16.0.
(6) Under nitrogen protection, the intermediate product 5 (1 mmol) was dissolved in DCM (5 mL), and TFA (5 mL) was added. The reaction was stirred at room temperature for 2 hours, and saturated NaHCO₃ solution was added to completely remove TFA. The organic phase was extracted with DCM (3×5 mL), and then sequentially washed with saturated brine (2 mL), dried with Na₂SO₄, and the oily residue was further dried in high vacuum to obtain a total product of crude deprotected amine, which was dissolved in N,N-dimethylformamide (5 mL), 2-[2-(tert-butoxycarbonylamino) ethoxy]ethoxy acetic acid (1 mmol) was added, and DIPEA (4 mmol) was added under stirring. 5 min later, HATU (1.1 mmol) was added. The reaction was stirred at room temperature for 18 hours. Water (50 mL) was added, and the reaction was extracted with EtOAc (3×25 mL). Then, the organic phase was sequentially washed with saturated brine (10 mL), dried with Na₂SO₄, filtered and concentrated in vacuum to obtain a crude product. The crude product was purified by column chromatography on silica gel (petroleum ether) to obtain a reactant 1. HRMS (ESI) m/z: [M+H]+ Calcd for C₄₀H₅₃N₅O₉S 779.36; Found 780.36181, as shown in FIG. 6.

### 2. Preparation of the GATA Transcription Factor Proteolysis Targeting Chimera Compound

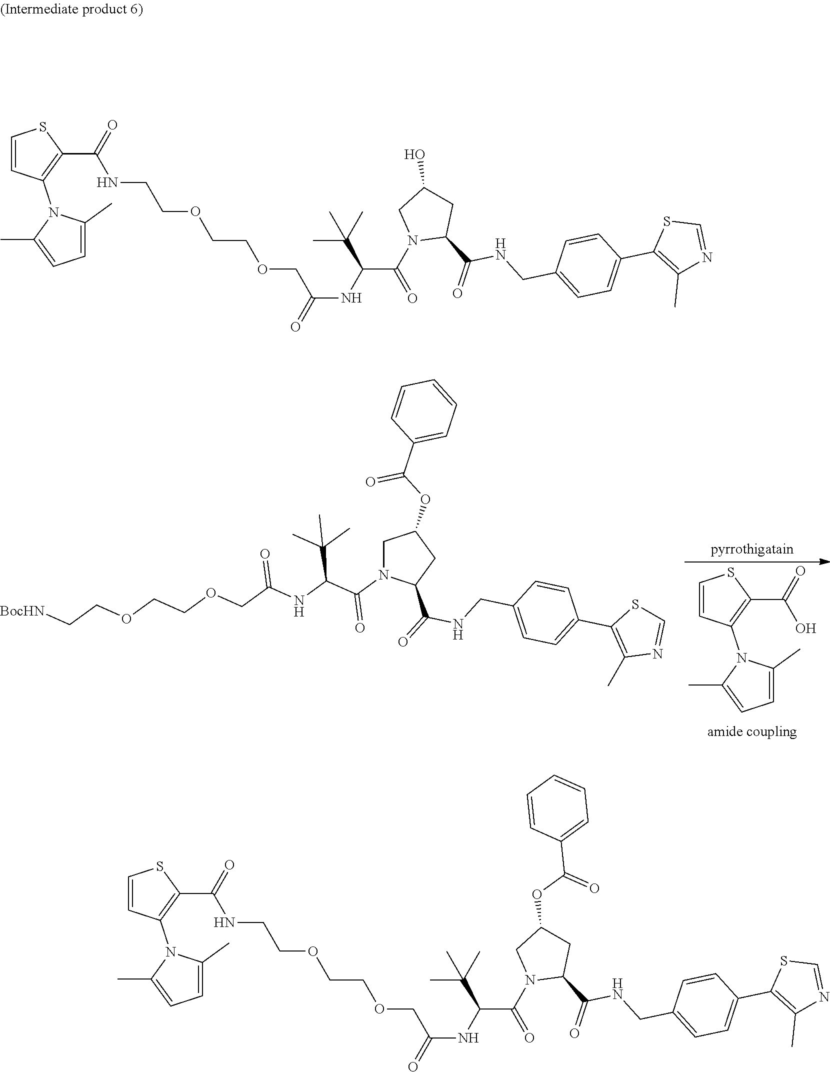

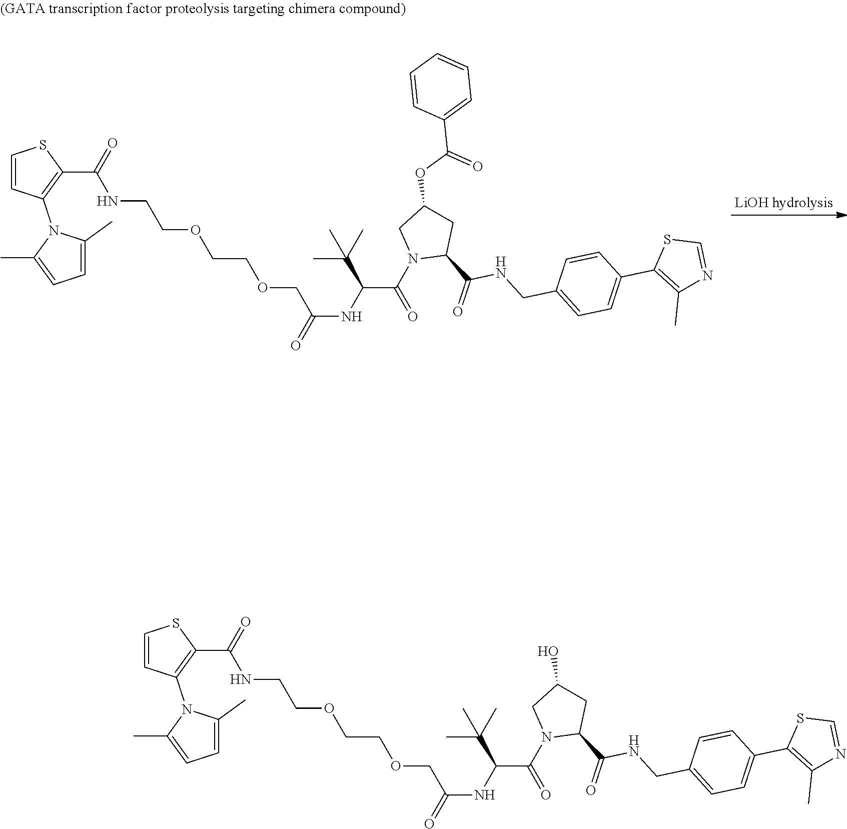 [SMILES: Cc1ncsc1-c1ccc(CNC(=O)[C@@H]2C[C@@H](OC(=O)c3ccccc3)CN2C(=O)[C@@H](NC(=O)COCCOCCNC(=O)c2sccc2-n2c(C)ccc2C)C(C)(C)C)cc1 ; Cc1ncsc1-c1ccc(CNC(=O)[C@@H]2C[C@@H](O)CN2C(=O)[C@@H](NC(=O)COCCOCCNC(=O)c2sccc2-n2c(C)ccc2C)C(C)(C)C)cc1]

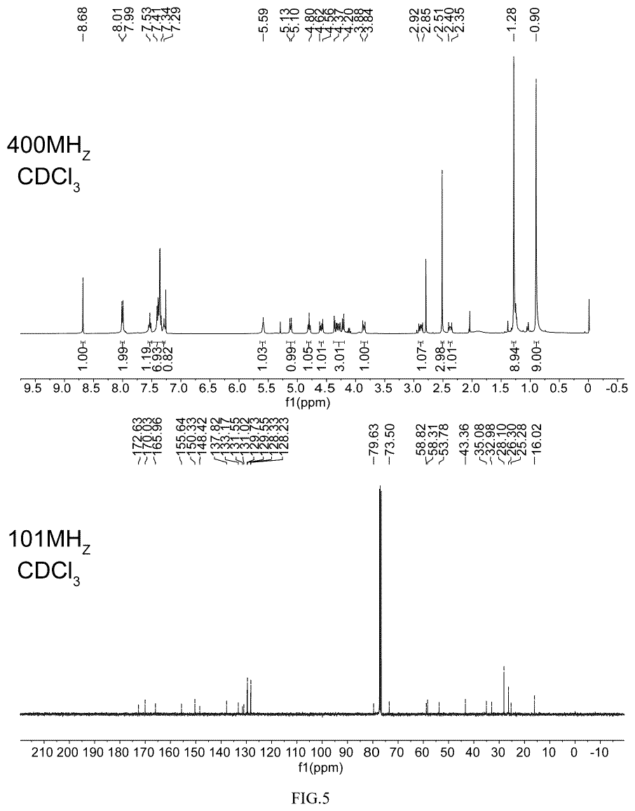

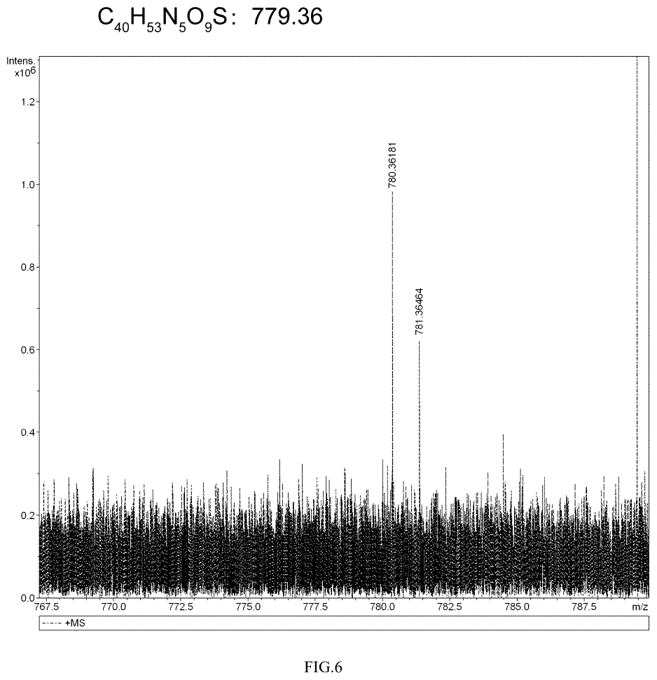

(1) Under nitrogen protection, the reactant 1 (1 mmol) was dissolved in DCM (5 ml), and TFA (5 ml) was added. The reaction was stirred at room temperature for 2 hours, and saturated NaHCO₃ solution was added to completely remove TFA. The organic phase was extracted with DCM (3×5 ml), and then sequentially washed with saturated brine (2 ml), dried with Na₂SO₄, and the oily residue was further dried in high vacuum to obtain a total product of crude deprotected amine, which was dissolved in N,N-dimethylformamide (5 ml), methyl 3-(2,5-dimethyl-1H-pyrrol-1-yl)-2-thiophenecarboxylate (1 mmol) was added, and DIPEA (4 mmol) was added under stirring. 5 min later, HATU (1.1 mmol) was added. The reaction was stirred at room temperature for 18 hours. Water (50 ml) was added, and the reaction was extracted with EtOAc (3×25 ml). Then, the organic phase was sequentially washed with saturated brine (10 ml), dried with Na₂SO₄, filtered and concentrated in vacuum to obtain a crude product. The crude product was purified by column chromatography on silica gel (petroleum ether) to obtain an intermediate product 6. HRMS (ESI) m/z: [M+Na]⁺ Calcd for C₄₆H₅₄N₆O₈S₂ 882.34; Found 905.33275, as shown in FIG. 7.
(2) Under nitrogen protection, the intermediate product 6 (1 mmol) was dissolved in THF (5 ml/mmol). At 0° C., a prepared lithium hydroxide aqueous solution was placed into the mixture, that is, lithium hydroxide (1.2 mmol) was dissolved in water (1.25 mmol), and stirred at 0° C. for 8 hours. After the mixture was neutralized with 1 mol/L of HCl, the organic phase was extracted with EtOAc (3×5 mL), then sequentially washed with saturated Na₂CO₃ (2 mL) and saturated brine (2 mL), dried over Na₂SO₄, filtered, and concentrated in vacuum to obtain a crude product. The crude product was purified by column chromatography on silica gel (petroleum ether) to obtain the GATA transcription factor proteolysis targeting chimera compound. The NMR results are shown in FIG. 8. ¹H NMR (400 MHz, CDCl₃) δ 8.67 (s, 1H), 7.54 (d, J=5.2 Hz, 1H), 7.50 (t, J=6.1 Hz, 1H), 7.38-7.28 (m, 5H), 6.93 (d, J=5.2 Hz, 1H), 5.95 (s, 2H), 5.25 (brs, 1H), 4.69 (t, J=8.0 Hz, 1H), 4.59-4.49 (m, 3H), 4.35 (dd, J=14.8, 5.2 Hz, 1H), 4.04-3.91 (m, 3H), 3.63-3.60 (m, 3H), 3.53-3.51 (m, 2H), 3.45-3.32 (m, 4H), 2.50 (s, 3H), 2.13-1.95 (m, 8H), 0.92 (s, 9H). ¹³C NMR (101 MHz, CDCl₃) δ 171.1, 170.8, 170.3, 160.5, 150.3, 148.4, 138.2, 135.6, 135.1, 131.7, 130.8, 129.5, 129.4, 128.4, 128.3, 128.2, 107.6, 71.2, 70.4, 70.1 (2C), 69.5, 58.5, 57.0, 56.7, 43.2, 39.5, 35.9, 35.2, 26.4, 16.1, 12.4.

### Embodiment 2

### 1. Experimental Materials

### 1.1 Cell Lines

Cells used in the present disclosure are all listed in the cell lines shown in Table 1:

TABLE 1
Cell lines

| Cell | | Cell |
|---|---|---|
| Name | Cell Type | Source |
| 4T1 | Mouse breast cancer cell lines | ATCC |
| T47D | Human breast cancer cell lines | ATCC |
| MCF7 | Human breast cancer cell lines | ATCC |
| 293T | Human kidney embryonic cell lines | ATCC |

### 1.2 Experimental Animals and Feeding-Related Materials

Balb/c mice used in the present disclosure were purchased from Jiangsu Huachuang Xinnuo Pharmaceutical Technology Co., Ltd., with the license number of SCXK (Su) 2020-0009; and all the experimental mice were raised in the SPF (specific pathogen free) grade laboratory of the animal experimental center of Nanjing Normal University. According to the feeding requirements, all the experimental mice were raised at a stocking density less than or equal to 5 per cage. The feeding was maintained at a temperature ranging from 20° C. to 25° C. and a humidity of around 50%, under the automatic light control (with 12 hours light/12 hours dark). The feed and bedding for mice were purchased from Qinglongshan Animal Breeding Farm in Jiangning District, Nanjing.

### 1.3 Experimental Reagents

### (1) Reagents Used in Cell Culture are Listed in Table 2: Cell Culture Reagents

TABLE 2
Cell Culture Reagents

| Reagent Name | Brand | Item No. |
|---|---|---|
| RPMI 1640 | WISENT | 350-000-CL |
| DMEM | WISENT | 319-005-CL |
| Fetal Bovine Serum (FBS) | WISENT | 086-150 |
| Phosphate buffer saline (PBS) | WISENT | 311-010-CL |
| Penicillin-streptomycin solution (PS) | WISENT | 450-201-EL |
| 0.25% TRYPSIN/EDTA | WISENT | 325-043-CL |
| Dimethyl sulfoxide (DMSO) | Amresco | 0457C072 |

### (2) Reagents Used in Biochemical Experiments are all Listed in Table 3

TABLE 3
Reagents for Biochemical and Molecular Experiments

| Name | Brand | Item No. |
|---|---|---|
| CCK8 | DOJINDO | CK04 |
| Polyethyleneimine | ALORICH | 408727 |
| Tryptone | OXOID | LP0042 |
| Yeast extract | OXOID | LP0021 |
| Agarose | BIOSHARP | 9012-36-6 |
| Agar powder | TSINGKE | TSJ001 |
| Ampicillin antibiotic (AMP) | Shanghai Biotech | 69-52-3 |
| Kana antibiotic (Kana) | Shanghai Biotech | 25389-94-0 |
| DpnI | NEB | R0176s |
| NotI | NEB | R3189L |
| EcoRI | NEB | R0101S |
| AgeI | NEB | R0552S |
| BamHI | NEB | R0136S |
| SacI | NEB | R0156S |
| XhoI | NEB | R0146S |
| T4 DNA Ligase | NEB | M0202L |
| High-fidelity PCR enzyme | Vazyme | P515-01 |
| DNA2000 | Vazyme | MD101-02 |
| DNA5000 | Vazyme | MD102-02 |
| Gel Red | BIOTIUM | 41003 |
| Plasmid mini-preparation kit | TIANGEN | DP103 |
| FastPure Gel DNA Extraction Mini Kit | Vazyme | DC301-01 |
| AxyPrep ™ RCR Clean kit | AXGEN | 15915KB1 |
| β-Mercaptoethanol | Bomei | QM7141 |
| RIPA lysis buffer (weak) | Beyotime | P0013D |
| Western and IP cell lysis buffer | Beyotime | P0013 |
| Three-color pre-stained protein marker | Epizyme | WJ103L |
| PMSF | Beyotime | ST505 |
| MG132 | Selleck | S2619 |

### (3) Antibodies Used in the Present Disclosure are all Listed in Table 4

TABLE 4
Antibodies

| Reagent Name | Brand | Item No. | Dilution ratio |
|---|---|---|---|
| GATA3 | Abcam | ab199428 | 1:2000 |
| Actin | Abclonal | AC006 | 1:5000 |
| HRP Goat anti-rabbit IgG | Abclonal | AS014 | 1:10000 |

### 1.4 Experimental Instruments

(1) Milli-Q ultrapure water system, American Millipore Company;
(2) UB-7 pH meter, Nanjing Henglian Biotechnology Co., Ltd.;
(3) 0.1-2.5 μL, 0.5-10 μL, 2-20 μL, 10-100 μL, 20-200 μL and 100-1000 μL pipettes, Eppendorf, Germany;
(4) XD-202 microscope, Nanjing Jiangnan Yongxin Optical Co., Ltd.;
(5) SW-CJ-IF type ultra-clean workbench, Suzhou Group Suzhou Antai Air Technology Co., Ltd.;
(6) Desktop refrigerated centrifuge, Eppendorf, Germany;
(7) DTH-100 metal bath, Nanjing Henglian Biotechnology Co., Ltd.;
(8) VOTTEX-2 vortex oscillator, Nanjing Henglian Biotechnology Co., Ltd.;
(9) HWS12 electric-heated thermostat water bath, Shanghai Yiheng Technology Co., Ltd.;
(10) Cell incubator, Thermo Fisher Scientific;
(11) StepOnePlus™ Real-Time PCR instrument, Bio-Rad, US;
(12) Nanodrop 2000, Thermo Scientific, US;
(13) BCD-328 EDPT refrigerator, Qingdao Haier Co., Ltd.;
(14) BCD-539WT low temperature refrigerator, Qingdao Haier Co., Ltd.;
(15) DW-86L626 ultra-low temperature refrigerator, Qingdao Haier Co., Ltd.;
(16) Vernier caliper, Nanjing Suzhou Measurement Instruments;
(17) Monolith NT.115, Nanotemper Technology;
(18) VE-180 vertical electrophoresis tank and transfer electrophoresis tank, Shanghai Tianneng Technology Co., Ltd.;
(19) UV-2000 UV analyzer, Shanghai Tianneng Technology Co., Ltd.;
(20) HVE-50 autoclave, HIRAYAMA Co., Japan;
(21) Thermo Mixer F1.5 thermomixer, Eppendorf, Germany;
(22) Leica DMI8 inverted fluorescence microscope, Beijing Zhongxian Hengye Instrument Co., Ltd.;
(23) Drying oven, Nanjing Henglian Biotechnology Co., Ltd.;
(24) MS-H280-pro magnetic stirrer, Shanghai Shengke Instrument Equipment Co., Ltd.;
(25) TY-80S decolorizing shaker, Puyang Scientific Instrument Research Institute;
(26) Flow cytometer, BD Biosciences;
(27) Microwave oven, Guangdong Galanz Group;
(28) DTH-100 metal bath, Nanjing Henglian Biotechnology Co., Ltd.;
(29) Scotsman ice machine, Scotsman Company;
(30) Fully automatic chemiluminescence spectrum analysis system, Shanghai Tianneng Technology Co., Ltd.
(31) 90° suction joint, vacuum dryer, splash-proof ball, thick-walled eggplant flask for rotary evaporator, liquid-adding funnel, flash chromatography column, chromatography cylinder, single-neck round-bottomed balloon flask, oblique three-neck balloon flask, straight three-neck balloon flask, solvent storage bottle, separatory funnel (with PTFE stopcock), vacuum gas distributor with double-row tube, Synthware.

### 1.5 Experimental Consumables

(1) 10 μL, 100 μL, and 1000 μL ordinary and DNase-/RNase-free pipette tips, Jiangsu Haimen Jiawei Glass Instrument Factory;
(2) 0.2 mL DNase-/RNase-free PCR tubes, 1.5 mL DNase-/RNase-free flat-cap centrifuge tubes, Axygen, US;
(3) 1.5 mL and 2 mL ordinary centrifuge tubes, Jiangsu Haimen Jiawei Glass Instrument Factory;
(4) 2 mL cell cryopreservation tubes (outward-spin), Corning;
(5) 100 mm and 60 mm cell culture dishes, Nanjing Shanqingbo Biotechnology Co., Ltd. (SORFA);
(6) 10 mL and 50 mL pipettes, Nanjing Shanqingbo Biotechnology Co., Ltd. (SORFA);
(7) Disposable PE gloves, Jiangsu Haimen Jiawei Instrument Factory;
(8) Disposable dust-free latex gloves, Jiangsu Haimen Jiawei Instrument Factory;
(9) Capillary tubes, Nanotemper Technologies;
(10) 15 mL and 50 mL centrifuge tubes, 6-well plates, 12-well plates, 24-well plates, 96-well plates, and cell culture dishes, Guangzhou Jiete Biofiltration Co., Ltd.;
(11) 0.22 μm and 0.45 μm disposable filter heads and PVDF membranes, Millipore, US;
(12) BRAUN surgical needles and syringes, Nanjing Boqiao Biological Co., Ltd.;
(13) Experimental filter paper, Xi'an Dingyu Filter Material Co., Ltd.
2. Experimental Methods

### 2.1 Cell Culture

4T1 mouse breast cancer cells, T47D human breast cancer cells, and MCF7 human breast cancer cells were cultured in 1640 medium supplemented with 10% serum and 1% penicillin/streptomycin at 37° C. in cell incubator containing 5% CO₂.

293T human renal embryo cells were cultured in DMEM medium supplemented with 10% serum and 1% penicillin/streptomycin at 37° C. in a cell incubator containing 5% CO₂.

### 2.2 Construction of Stably Transfected Cell Lines of Lentiviral System

(1) Construction of pCDH-GFP-Puro-GATA3-WT Eukaryotic Expression Plasmid.

### 1) Design, Synthesis and Sequencing of Primers

The primers were designed on the NCBI official website based on the specific gene sequence number, and subjected to blast alignment of primer sequences on the NCBI website to ensure the specificity of the template. The synthesis and sequencing services for the primers were provided by Suzhou Jinweizhi Biotechnology Co., Ltd. and Nanjing Qingke Biotechnology Co., Ltd.

### Primer Sequence:

Forward: 5 ‘TA GCTAGC ATG TAC CCA TAC GAC GTC CCA GAC TAC GCT ATGGAGGTGACGGCGGACCAGCCG 3’ (SEQ NO: 1)
Reverse: 5 ‘T GCGGCCGC CTA ACCCATGGCGGTGACCATGCT 3’ (SEQ NO: 2)

Taking the GATA3 WT plasmid as template, the PCR program was set up according to the following system:

### PCR Reaction System:

| Reagents | Volume | |
|---|---|---|
| DNA template | 1 μL | (0.1-1 μg) |
| Forward primer | 0.5 μL | (10 nmol) |
| Reverse primer | 0.5 μL | (10 nmol) |
| 2 × PCR Master Mix | 5 | μL |
| ddH₂O | 3 | μL |
| Total | 10 | μL |

### PCR Reaction Procedure:

| Stage | Temperature and time | |
|---|---|---|
| Pre-denaturation | 95° C., | 3 min |
| Denaturation | 95° C., | 30 sec |
| Annealing | 56-60° C., | 30 sec |
| Extension | 72° C., | 30 sec/kb |
| Post-extension | 72° C., | 10 min |
| Stand | 4° C., | unlimited |

2) The PCR recovery product and the target vector (pCDH-CMV-MCS-EF1-GFP-Puro vector, YouBio VT8070) were subjected to enzyme digestion. The PCR recovery product and the target vector were respectively mixed in EP tubes according to the enzyme digestion system, and placed into a 37° C. thermostat incubator. After 1-2 hours of enzyme digestion, the reaction mixture was subjected to DNA gel electrophoresis and purified using Axgen Gel Extraction kit.

### Enzyme Digestion System:

| Reagents | Volume | |
|---|---|---|
| Vectors | 1 μL (1 μg/μL) | |
| Enzyme 1 (Nhe I) | 0.5 | μL |
| Enzyme 2 (Not1) | 0.5 | μL |
| 10 × NEB CutSmart buffer | 1 | μL |
| ddH₂O | 7 | μL |
| Total | 10 | μL |

3) The purified target fragment DNA and vector were mixed in an EP tube according to the ligation system. After 2 hours of ligation at room temperature, the ligation product was transformed into DH5a competent cells.

### Ligation System:

| Reagents | Volume | |
|---|---|---|
| Vectors | 1 μL (50 ng/μL) | |
| DNA fragment | 8 | μL |
| T4 Ligase | 0.5 | μL |
| 10 × T4 Ligase buffer | 1 | μL |
| ddH₂O | 4.5 | μL |
| Total | 15 | μL |

4) After transformation, the reaction mixture was cultured. Plasmids were extracted using a plasmid extraction kit, and collected for sequencing identification. The correctly sequenced plasmids were transformed and further cultured in large scale. Plasmids were extracted for later use to obtain the pCDH-GFP-Puro-GATA3-WT target plasmids.
(2) HEK-293T cells were cultured in a 10-cm culture dish. When the cells grew to 70-90%, the constructed pCDH-GFP-Puro-GATA3-WT target plasmids, together with the pMDLg/pRRE, pRSV-Rev, and VSVG packaging plasmids purchased from YouBio were transformed into the 293T cells.
(3) 48 hours after the transfection, the supernatant virus liquor was collected and filtered with a 0.45-μm filter membrane (which could be stored at −80° C. for later use).
(4) The cells to be infected were spread into a 6-well plate or a 10-cm culture dish. When the cell density reached 30%-40%, the original medium was discarded and replenished with fresh medium. A concentrated virus solution was added, while PB was added at a ratio of 1:1000-1:1500. The mixture was continued to culture in the incubator.
(5) 24 hours after the infection, the medium was refreshed. According to different cells, appropriate concentrations of puromycin were added to screen surviving cells. The cells were continued to culture, until a stably passaged cell line was screened, that is, 4T1 mouse breast cancer cells that stably and highly express GATA3 (4T1-GATA3-WT cells).

### 2.3 Western Blotting

### 2.3.1 Extraction of Protein (Operation on Ice)

(1) The cultured cells were taken out of the incubator, the medium was discarded, and the cells were washed with PBS. After digestion of the cells with trypsin at 37° C., a complete medium was added to stop the digestion process, and then transferred to a 1.5 mL centrifuge tube. The mixture was centrifuged at 1000 g for 5 min. The supernatant was discarded, and the cell pellets were collected and washed with PBS for later use;
(2) A RIPA lysis buffer was formulated so that PMSF was formulated to a concentration of 1 mg/ml. The cell precipitate was resuspended, and lysed on ice for 15 min.
(3) After the lysis, the mixture was centrifuged at 13,000 rpm at 4° C. for 10 min. The supernatant was transferred to a new 1.5 mL centrifuge tube, an appropriate amount of 5× Loading buffer was added. The mixture was mixed evenly and heated in a 95° C. metal bath for 15 min. At the end of heating, the mixture was subjected to flash centrifugation for 5 min, and stored at −20° C. for later use.

### 2.3.2 SDS-PAGE Gel Electrophoresis

The formulae of separating gel and stacking gel are shown in Table 5.

TABLE 5
Formulae of Separating Gel and Stacking Gel

| | | | Stacking |
|---|---|---|---|
| | Separating gel (5 mL) | | gel (3 mL) |
| Names of Various Components | 10% | 15% | 5% |
| ddH₂O | 1.9 | 1.1 | 2.1 |
| 30% Acrylamide | 1.7 | 2.5 | 0.5 |
| 1.5M Tris-HCl (pH 8.8) | 1.3 | 1.3 | — |
| 1.0M Tris-HCl (pH 6.8) | — | — | 0.38 |
| 10% SDS | 0.05 | 0.05 | 0.03 |
| 10% AP | 0.05 | 0.05 | 0.03 |
| TEMED | 0.002 | 0.002 | 0.003 |

(1) The separating gels with different concentrations were selected according to the size of the protein to be detected;
(2) An appropriate SDS-PAGE separating gel was formulated according to Table 5, mixed evenly, and injected between two clean glass plates with edge thickness of 1.0 mm. A thin layer of isopropyl alcohol was added on top and stood at room temperature for 30 min;
(3) After the lower layer of gel was completely solidified, the upper layer of isopropyl alcohol was poured away. The plates were invertedly placed in a 37° C. oven for 5 min. A stacking gel was slowly added, and a loading comb was inserted. The plates were left to stand at room temperature for 30 min;
(4) A 1×SDS running buffer was prepared. The gel was fixed on the electrophoresis device, and an electrophoresis buffer was poured into the upper and lower tanks. The loading comb was carefully removed to check whether the loading holes were vertical;
(5) The protein sample to be used was taken out of the −20° C. refrigerator, heated in a 95° C. metal bath for 5 min, and centrifuged at 12,000 rpm for 5 min;
(6) The gel electrophoresis instrument was powered and the voltage was adjusted to 120V. After the protein sample was pressed into a line on the stacking gel, the voltage was adjusted to 180V to continue the electrophoresis. At the end of electrophoresis, the gel was taken out of the glass plate. And the gel, a pre-cooled transfer buffer, four sheets of filter paper with the same size as the gel plate, and a PVDF membrane with the same size as the gel which had been activated in methanol were laid and clamped in a sequence of the black side of the transfer clamp, sponge pad, two layers of filter paper, gel, PVDF membrane, two layers of filter paper, sponge pad and transparent side of the transfer clamp (no bubbles should be generated during this process);
(7) The transfer well plate was placed according to the positive and negative directions into the transfer tank, which was then filled with a pre-cooled transfer fluid. The entire transfer electrophoresis tank was placed into a foam box containing an ice-water mixture, and powered with a constant current of 200 mA for 2 hours;
(8) At the end of transfer, the PVDF membrane was rinsed twice with TBST solution, and then soaked in 5% skim milk or 5% BSA for blocking with slow shaking in a shaker at room temperature for 1 hour;
(9) At the end of blocking, the PVDF membrane was rinsed 3 times with TBST to remove the residual blocking solution. Then, the PVDF membrane was cut to an appropriate width according to the sizes of the protein marker and target protein band, and incubated with the primary antibody with slow shaking in a shaker at 4° C. overnight;
(10) On the next day, the PVDF membrane was taken out and placed in a membrane washing box, rinsed 5 times with TBST for 3 min each time. The washed PVDF membrane was transferred to the proportionally formulated corresponding secondary antibody, and incubated at room temperature for 1 hour;
(11) The PVDF membrane in the membrane washing box was further rinsed with TBST 5 times for 3 min each time;
(12) Development. According to the size of the PVDF membrane, two developing substrates were equivolumetrically mixed evenly at a ratio of 1:1 to prepare an appropriate amount of ECL luminescent liquid mixture. First, the PBST was removed from the PVDF membrane, and the luminescent liquid was added. Then, the membrane was placed into the fully automatic chemiluminescence spectrum analysis system for detection.

### 2.4 IC₅₀ Detection

(1) The cells in good growth status were digested into single cells with 0.25% trypsin. After counting with a hemocytometer, the cells were placed in a 96-well plate at 3000 cells/well, and cultured in a 37° C., 5% CO₂ cell incubator;
(2) After complete attachment and spread, the cells were divided into dosing treatment experimental groups with different concentrations and control group treated in equal proportions according to the dosing volumes of the experimental groups. 3 replicate wells were set for each group;
(3) After 48 hours, a complete medium and CCK-8 reagent were mixed evenly at a ratio of 9:1 as needed. At the same time, the medium in the well was discarded, and 100 μL of mixed solution was added to each well. The well plate was cultured in the incubator for additional 2 hours. At the end of incubation, the mixed liquor was transferred into a new 96-well plate in order (note: no bubbles should be generated in the wells to avoid affecting the OD value), and the absorbance value at 450 nm was measured with a microplate reader;

$${IC}_{50} of cells = ( A value of experimental group - A value of calibration group ) / ( A value of control group - A value of calibration group ) * 100 \% . \qquad ( 4 )$$

The above experiment was repeated three times to obtain an average value.

### 2.5 Cell Proliferation

(1) Cells in logarithmic growth phase were digested with 0.25% trypsin to form a suspension of single cells, which were counted on a hemocytometer. The cells were placed in a 96-well plate at 1500 cells/well, and cultured in a 37° C., 5% CO₂ cell incubator;
(2) On the next day, the fully adherent and spread cells were subjected to concentration gradient treatment or no-drug treatment. Each group was set up with 3 duplicate wells, and the mixed liquor was added to the corresponding well plate at a volume of 100 μL per well. The well plate was cultured in the incubator for additional 2 hours, and the absorbance value at 450 nm was measured with a microplate reader. At that time, the measured value was the absorbance on Day 0;
(3) Then, based on the time on Day 0 as baseline, the old medium in the 96 wells was discarded every 24 hours and replenished with 100 μL of the formulated CCK-8 working solution to each well, the well plate was cultured in the incubator for additional 2 hours, and the cells were measured with a microplate reader for their absorbance value at 450 nm.

### 2.6 Detection of Cell Apoptosis

MCF7 and T47D cells were spread into a 10-cm cell dish. When the cell density reached about 70%, different concentrations of the GATA transcription factor proteolysis targeting chimera compound and corresponding volumes of DMSO as negative controls were added and cultured for 24 hours. The cells in the 10-cm dish were carefully collected into a centrifuge tube, washed with PBS, and digested with EDTA-free trypsin until the cells could be gently blown down with a pipette or pipette tip. All the adherent cells were slowly blown down, and centrifuged at around 1000 rpm for 5 min to precipitate the cells. The cells were resuspended in a pre-cooled PBS at 4° C., and then re-centrifuged to precipitate the cells. The supernatant was carefully absorbed away. A negative control group of cells was set, that is, those with no Annexin V/FITC and propidium iodide solution (PI), for adjusting the voltage. A single staining tube: only Annexin V/FITC and PI were added as a single positive control group for adjusting the compensation. Detection tube: 10× Binding Buffer was diluted with deionized water to 1 at a ratio of 1:9. The cells were resuspended and adjusted to a concentration of 1-5×10⁶/ml. 5 μl of Annexin V/FITC was added. The mixture was mixed evenly and incubated at room temperature in the dark for 5 min. Again, additional 5 μl of propidium iodide solution (PI) was added, and the flow cytometric tube was supplemented with PBS. Then, a flow cytometric detection was carried out immediately.

### 2.7 Mouse Tumor Model and Administration

Construction of breast cancer cells stably expressing the GATA3 (i.e., 4T1-GATA3-WT cells) by syngeneic graft lentivirus system in mouse models: 4-5-week-old commercially available Balb/c female mice were labeled by toe clipping method. Each mouse was injected with 1×10⁵ 4T1-GATA3-WT cells at the third breast pad on the left. After the formation of tumor (with a volume of about 100 mm³), the GATA transcription factor proteolysis targeting chimera compound was dissolved in 20% β-cyclodextrin, and administrated to treat the tumor at a single dose of 25 mg/kg/mouse and 50 mg/kg/mouse once every two days through the tail vein, with normal saline as control. During the trial, the mice were monitored for the toxicity signs of the GATA transcription factor proteolysis targeting chimera compound.

### 2.8 Evaluation of the Activity of Experimental Compounds in Mouse Tumor Models

The bedding, feeding, and drinking water for mice were changed once or twice a week. Starting from the injection of 4T1-GATA3-WT cells, each mouse was palpated every 3 days to check whether nodule-like touch, i.e. breast tumor, would appear in the mouse breasts. The time of first touching the breast tumor was recorded, and the mice began to be weighed every 2 days and recorded for the length and width of tumor. According to the formula: tumor volume=(length×width²)/2, the tumor volume was calculated and recorded for each mouse. The length and width of tumor were both measured with vernier calipers. After the tumor volume reached 100 mm³, the tumor-bearing mice were randomly grouped and injected through the tail vein with normal saline and different concentration gradient of drugs, that is, 25 mg/kg/mouse and 50 mg/kg/mouse. When the tumor volume of mice in the normal saline group reached 1500 mm³, all the mice in the experimental groups and the control group were euthanized. At the same time, the breast tumor tissues of the tumor-bearing mice were washed once with PBS, wiped clean with absorbent paper, wrapped in tin foil, labeled with the mouse number, treatment method and date, and stored at-80 degrees.

### 2.9 Protein Expression and Microscale Thermophoresis (MST)

GATA3 was cloned into the pEGFP-N2-Vector vector (YouBio VT1111), and transfected into 293T to overexpress the GATA3 protein fused with EGFP. 48 hours after the transfection, the cell pellets were collected, lysed on ice to extract the protein, and measured by Coomassie Brilliant Blue method for the protein concentration. Then, the fluorescence intensity of the GATA3 protein fused with EGFP was pre-detected using the Monolith NT.115 software, and according to the detected fluorescence intensity, the protein was diluted in PBST to an appropriate concentration. The EGFP-labeled protein was mixed with equivalent volume of 16 unlabeled compounds in a series of different concentrations and incubated at room temperature. After 5 min, the sample was placed into a capillary tube, directly detected at 25° C., and the binding affinity between GATA3 and the compound was calculated. The data was analyzed using the MO. Affinity Analysis v.2.3 software to obtain the Kd values.

### 3. Experimental Results

3.1 The identification of the in vitro binding capacity between the GATA transcription factor proteolysis targeting chimera compound and the GATA3 protein was carried out by the above-mentioned protein expression and microscale thermophoresis experiment (MST) method.

A full-length GATA3 gene was constructed on the pEGFP-N2-Vector vector, and the GATA3 protein fused with EGFP was highly expressed by 293T cells. The cells were lysed and the supernatant was taken. According to the fluorescence intensity of the fusion protein expression, the protein was adjusted with PBST to an appropriate concentration, and the in vitro binding force between the unlabeled GATA transcription factor proteolysis targeting chimera compound and the EGFP-labeled GATA3 protein was detected by microscale thermophoresis (MST). As shown in FIG. 9, the binding force therebetween reaches a nanomolar level, indicating that the GATA transcription factor proteolysis targeting chimera compound has strong binding force with GATA3. At the same time, taking the in vitro binding force between the GATA3 ligand represented by formula II and the EGFP-labeled GATA3 protein at micromolar level as control, the compound of the present disclosure has more than 30,000 times higher than that of the GATA3 ligand (FIG. 9).
3.2 The GATA transcription factor proteolysis targeting chimera compound is a GATA3 degrader that effectively degrades the GATA3 protein, as demonstrated by the above Western blotting method.

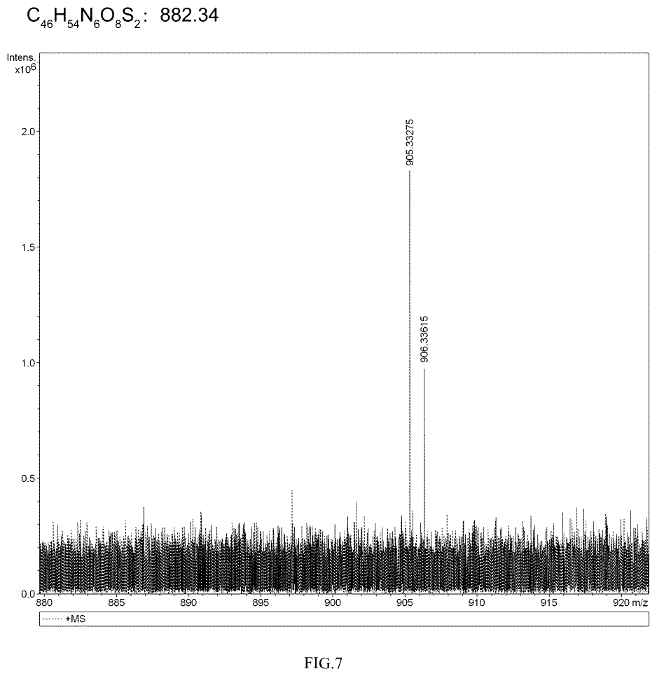

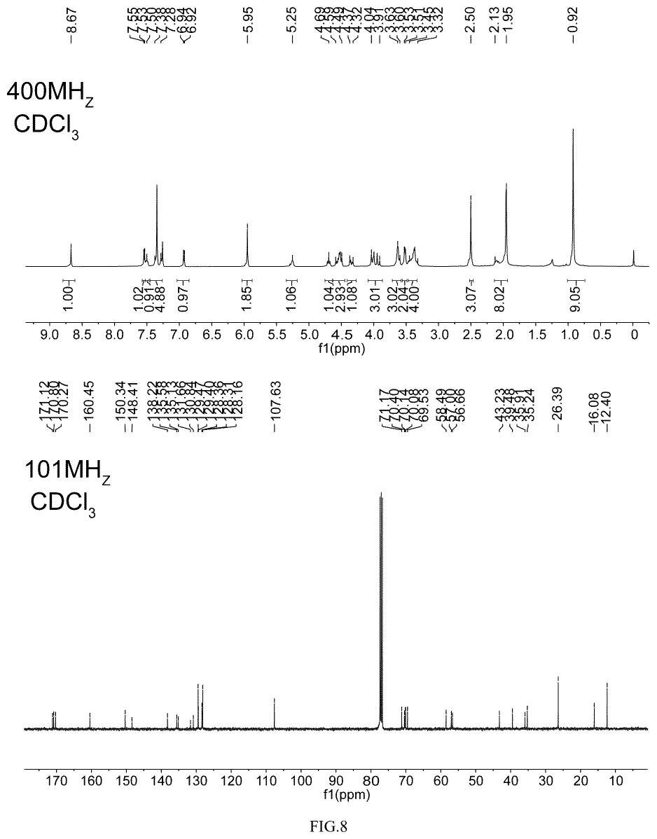

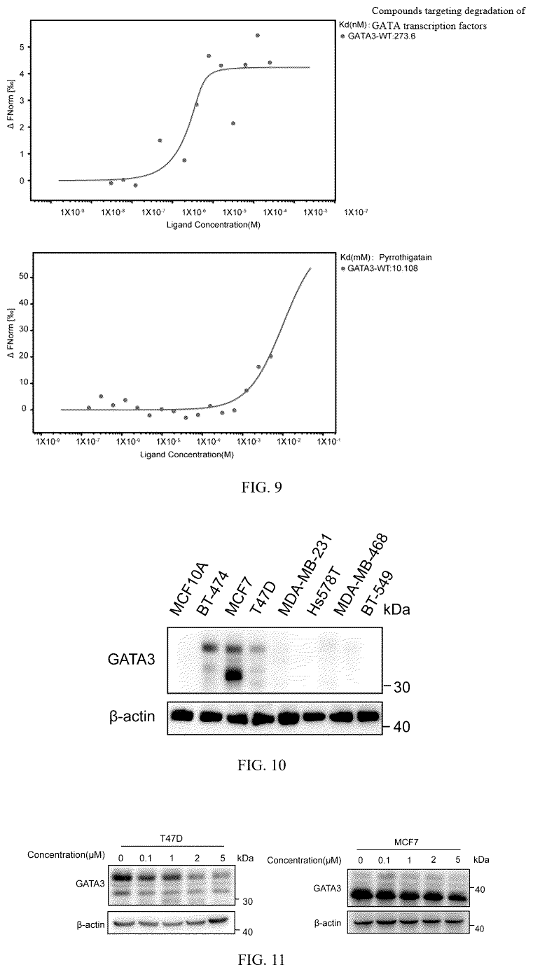

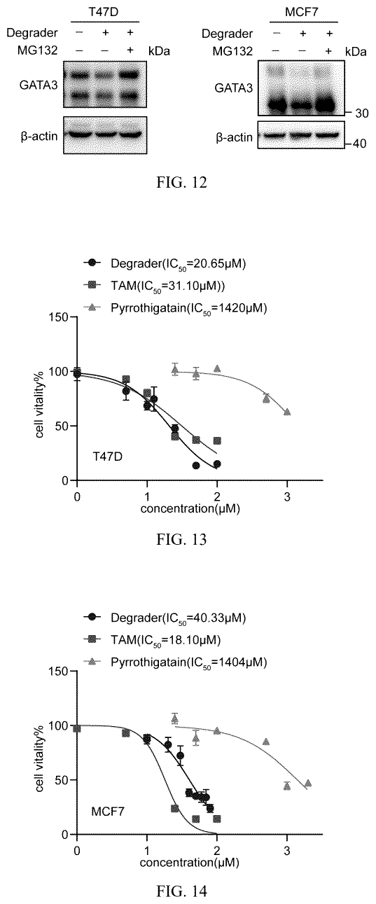

Using breast cancer cell lines, various types of cells were detected for the GATA3 expression profile at the protein level. As shown in FIG. 10, BT474, T47D, and MCF7 have high GATA3 expression. Among them, T47D and BT474 have the same GATA3 expression pattern, so only T47D and MCF7 were selected as model cells for subsequent verification. After the T47D and MCF7 cells were treated with DMSO and GATA transcription factor proteolysis targeting chimera compound with different concentration gradients for 24 hours, respectively, the cell pellets were collected and lysed to extract the protein supernatant. After measuring the protein concentration, the cells were detected for the GATA3 protein content, with the results shown in FIG. 11. With the increasing drug concentration, the ability of the GATA transcription factor proteolysis targeting chimera compound to degrade the GATA3 protein increases, indicating that the degradation activity of the GATA transcription factor proteolysis targeting chimera compound depends on the drug concentration. In addition, the mechanism of the GATA transcription factor proteolysis targeting chimera compounds inhibiting GATA3 is based on the intracellular ubiquitinated enzymosome degradation pathway. Therefore, the addition of the proteasome inhibitor MG132 can also restore the degradation of GATA3 by the GATA transcription factor proteolysis targeting chimera compound, as shown in FIG. 12.
3.3 The anti-breast cancer efficacy of the GATA transcription factor proteolysis targeting chimera compound in vitro was significantly improved, as demonstrated by the above IC₅₀ detection, cell proliferation, and cell apoptosis methods.

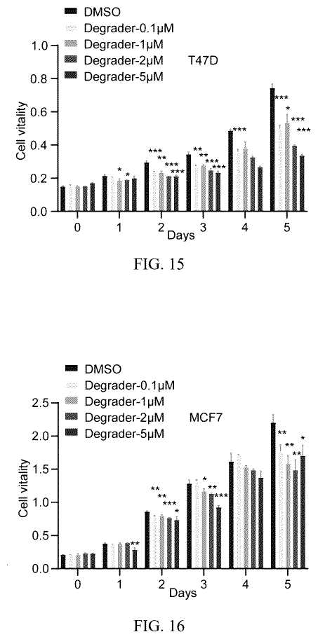

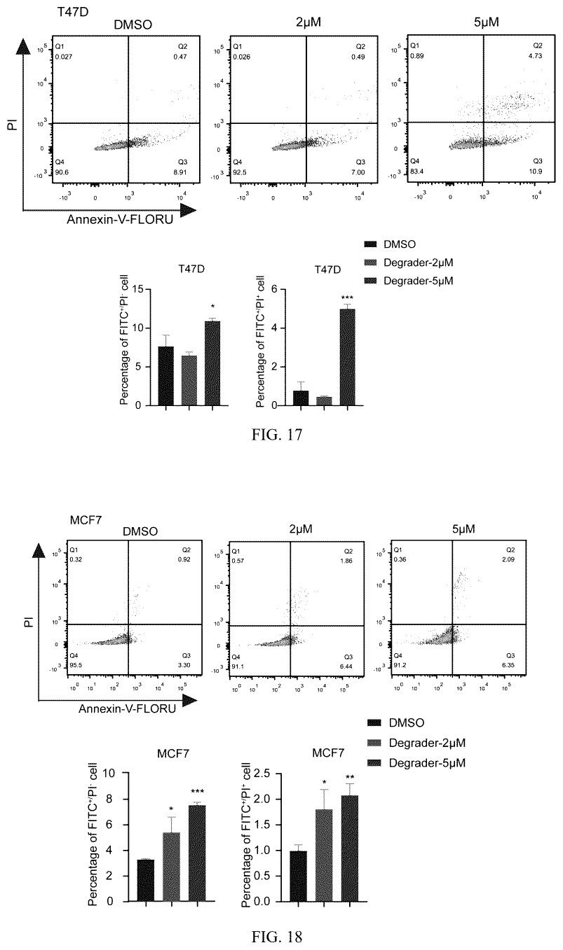

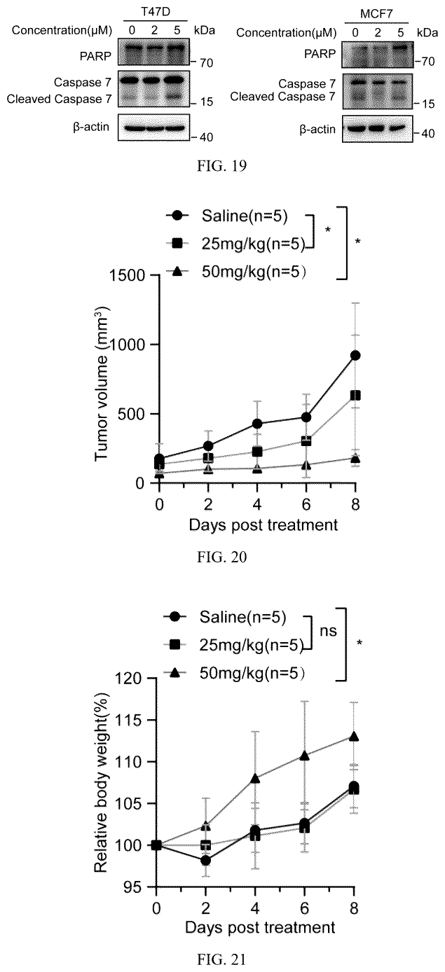

The killing ability of the GATA transcription factor proteolysis targeting chimera compound with different concentrations on MCF7 and T47D was tested, with Pyrrothigatain and Tamoxifen as respective negative and positive controls. The results are shown in FIGS. 13 and 14. Compared with the GATA3 ligand Pyrrothigatain represented by formula II alone, the GATA transcription factor proteolysis targeting chimera compound has significant killing ability against two types of cancer cells with efficacy increased by 40-70 times. The killing performance of the GATA transcription factor proteolysis targeting chimera compound against MCF7 is comparable to those of classic therapeutic drugs for ER-positive breast cancers such as Tamoxifen, while the effect of the GATA transcription factor proteolysis targeting chimera compound on T47D is even higher than that of Tamoxifen. The above results indicate that the GATA transcription factor proteolysis targeting chimera compounds can directly kill cancer cells such as MCF7 and T47D. Next, the effect of the GATA transcription factor proteolysis targeting chimera compound with different concentration gradients on the proliferation and apoptosis abilities of MCF7 and T47D were tested, with the results shown in FIGS. 15-19. The results show that the GATA transcription factor proteolysis targeting chimera compound significantly inhibit the proliferation ability of MCF7 and T47D and promote the cell apoptosis, while the efficacy depends on the concentration of the GATA transcription factor proteolysis targeting chimera compound.

### 3.4 Effect of GATA Transcription Factor Proteolysis Targeting Chimera Compound on the Breast Tumor Growth in Mice

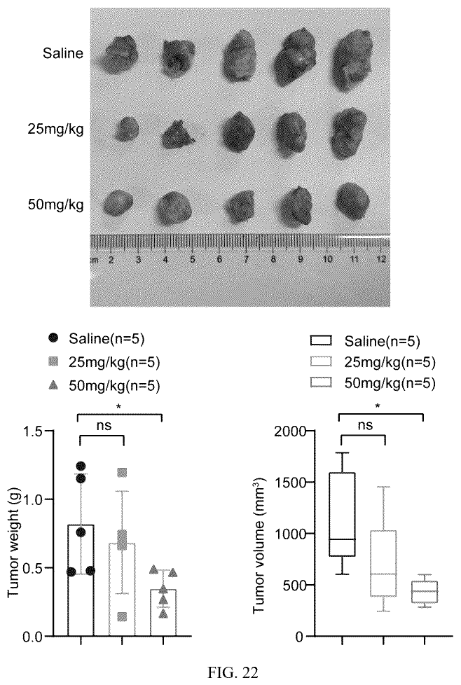

A number of 4T1 mouse breast cancer cells which stably and highly expressed GATA3 (4T1-GATA3-WT cells) were injected into the fat pad of Balb/c mice to construct a syngeneic graft tumor model. After the tumor was formed in mice and reached a volume of 100 mm³, the mice were randomly grouped and injected through tail vein with 25 mg/kg/mouse and 50 mg/kg/mouse of the GATA transcription factor proteolysis targeting chimera compound every two days. At the same time, the weight was recorded for each mouse. Among them, normal saline was used as the control. As shown in FIGS. 20-21, the results show that compared with the normal saline group, the tumor volumes of the mice in the low- and high-concentration groups were significantly inhibited compared with that of the control group in the absence of obvious toxicity and side effects. Among them, therapeutic effect on the tumor volume of the mice in the high-concentration group was significantly higher than that of the lower-concentration group. When the tumor volume of the mice in the control group grew to about 1500 mm³, all the mice in the experimental groups and the control group were euthanized. At the same time, the breast tumor tissue of the tumor-bearing mice was peeled off and weighed. The volume of the peeled tumor was measured with a vernier caliper and recorded, with the results shown in FIG. 22. The above results all show that the GATA transcription factor proteolysis targeting chimera compound significantly inhibits the tumor growth in mice and can be used in the preparation of a drug for treating breast cancer.

1. A GATA transcription factor proteolysis targeting chimera compound and pharmaceutically acceptable salts thereof, wherein the compound has a structure represented by formula (I):

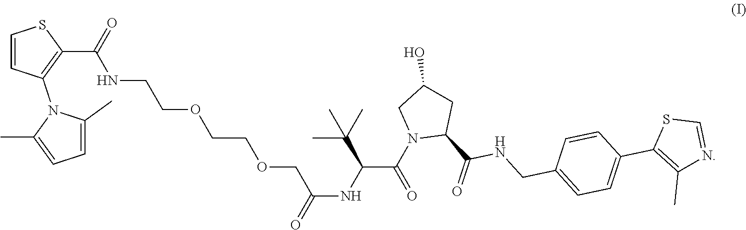 [SMILES: Cc1ncsc1-c1ccc(CNC(=O)[C@@H]2C[C@@H](O)CN2C(=O)[C@@H](NC(=O)COCCOCCNC(=O)c2sccc2-n2c(C)ccc2C)C(C)(C)C)cc1]

2. The GATA transcription factor proteolysis targeting chimera compound and pharmaceutically acceptable salts thereof according to claim 1, wherein the compound contains three core component compounds having structures represented by formula (II), formula (III), and formula (IV), respectively:
GATA protein binding compound (II): 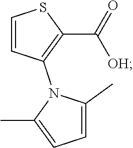
Linker (III): 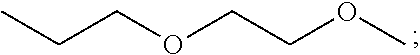
VHL ligand (IV):

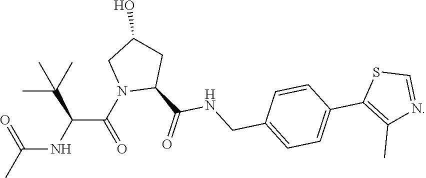 [SMILES: CC(=O)N[C@H](C(=O)N1C[C@H](O)C[C@H]1C(=O)NCc1ccc(-c2scnc2C)cc1)C(C)(C)C]

3. A method for preparing the GATA transcription factor proteolysis targeting chimera compound and pharmaceutically acceptable salts thereof according to claim 2, wherein the method has a reaction process of:

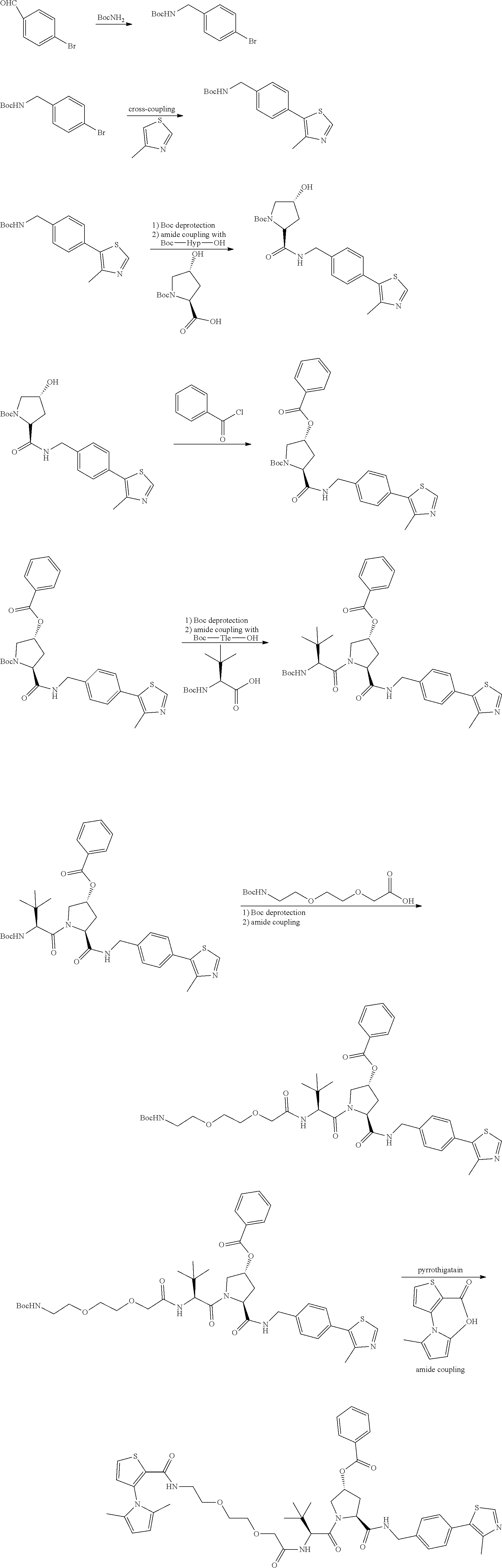

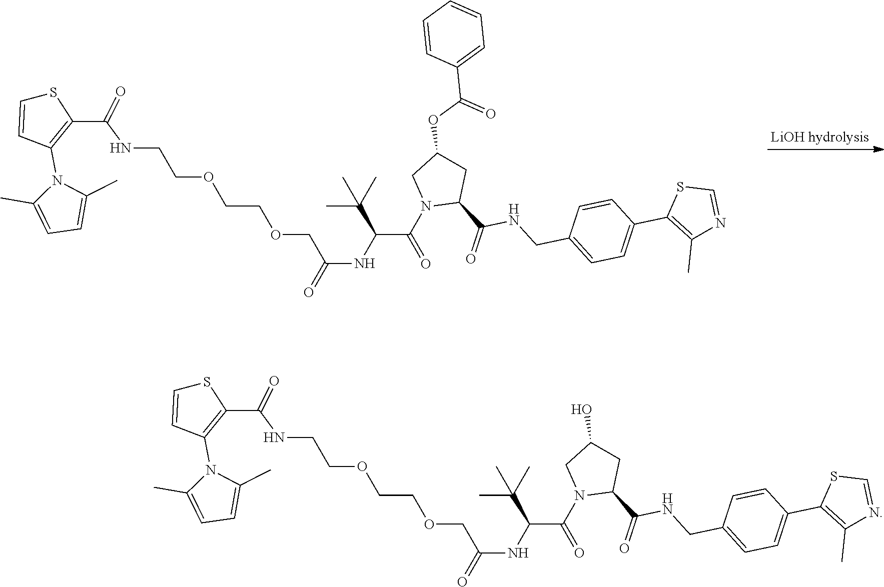 [SMILES: Cc1ncsc1-c1ccc(CNC(=O)[C@@H]2C[C@@H](OC(=O)c3ccccc3)CN2C(=O)[C@@H](NC(=O)COCCOCCNC(=O)c2sccc2-n2c(C)ccc2C)C(C)(C)C)cc1 ; Cc1ncsc1-c1ccc(CNC(=O)[C@@H]2C[C@@H](O)CN2C(=O)[C@@H](NC(=O)COCCOCCNC(=O)c2sccc2-n2c(C)ccc2C)C(C)(C)C)cc1]

4. The method according to claim 3, wherein the method comprises the following steps:
(1) slowly and sequentially dropwise adding Et₃SiH and TFA to tert-butyl carbamate and 4-bromobenzaldehyde in a solvent, stirring to react, then quenching the reaction, then extracting, washing, drying, filtering, and concentrating in vacuum to obtain a crude product, and isolating and purifying the crude product to obtain an intermediate product 1; 10
(2) dissolving the intermediate product 1, 4-methylthiazole, Pd(OAc)₂, and K₂CO₃ in N,N-dimethylacetamide, heating to react, then cooling to room temperature, diluting, then extracting, washing, drying, filtering, and concentrating in vacuum to obtain a crude product, and isolating and purifying the crude product to obtain an intermediate product 2;
(3) dissolving the intermediate product 2 in DCM, adding TFA, stirring to react, then extracting, washing, drying to obtain a crude deprotected amine, and dissolving the crude deprotected amine in N,N-dimethylformamide, adding boc-L-hydroxyproline, then adding DIPEA and HATU sequentially while stirring to react, then adding water, extracting, washing, drying, filtering, and concentrating in vacuum to obtain a crude product, and isolating and purifying the crude product to obtain an intermediate product 3;
(4) dissolving the intermediate product 3, benzoyl chloride and DMAP in DCM, and adding TEA under an ice bath condition, then stirring to react, adding H₂O, extracting, washing, drying, filtering, and concentrating in vacuum to obtain a crude product, and isolating and purifying the crude product to obtain an intermediate product 4;
(5) dissolving the intermediate product 4 in DCM, adding TFA, stirring to react, extracting, washing, and drying to obtain a crude deprotected amine, dissolving the crude deprotected amine in N,N-dimethylformamide, adding boc-L-tert-leucine, then adding DIPEA and HATU sequentially while stirring to react, then adding water, extracting, washing, drying, filtering, and concentrating in vacuum to obtain a crude product, and isolating and purifying the crude product to obtain an intermediate product 5;
(6) dissolving the intermediate product 5 in DCM, adding TFA, stirring to react, extracting, washing, and drying to obtain a crude deprotected amine, dissolving the crude deprotected amine in N,N-dimethylformamide, adding 2-[2-(tert-butoxycarbonylamino) ethoxy]ethoxy acetic acid, then adding DIPEA and HATU sequentially while stirring to react, then adding water, extracting, washing, drying, filtering, and concentrating in vacuum to obtain a crude product, and isolating and purifying the crude product to obtain a reactant 1;
(7) dissolving the reactant 1 in DCM, adding TFA, stirring to react, extracting, washing, and drying to obtain a crude deprotected amine, dissolving the crude deprotected amine in N,N-dimethylformamide, adding methyl 3-(2,5-dimethyl-1H-pyrrol-1-yl)-2-thiophene carboxylate, then adding DIPEA and HATU sequentially while stirring to react, then adding water, extracting, washing, drying, filtering, and concentrating in vacuum to obtain a crude product, and isolating and purifying the crude product to obtain an intermediate product 6; and
(8) dissolving the intermediate product 6 in THE, placing a prepared aqueous solution of lithium hydroxide into the mixture under an ice bath condition, stirring to react, after neutralizing the mixture, extracting, drying, filtering, and concentrating in vacuum to obtain a crude product, and isolating and purifying the crude product to obtain the GATA transcription factor proteolysis targeting chimera compound.

5. A use of the compound and pharmaceutically acceptable salts thereof according to claim 1 in preparation of a drug for degrading a GATA transcription factor or a drug for treating GATA transcription factor abnormalities.

6. The use according to claim 5, wherein the use of the compound and pharmaceutically acceptable salts thereof is in preparation of a degradation agent for degrading a GATA3 protein.

7. A use of the compound and pharmaceutically acceptable salts thereof according to claim 1 in the preparation of a drug for treating cancer.

8. The use according to claim 7, wherein the use of the compound and pharmaceutically acceptable salts thereof is in preparation of a drug for treating breast cancer.

9. A method for degrading a GATA transcription factor or treating GATA transcription factor abnormalities in a subject in need thereof, comprising:
administering to the subject a therapeutically-effective amount of the compound according to claim 1.

10. A method for degrading a GATA3 protein in a subject in need thereof, comprising:
administering to the subject a therapeutically-effective amount of the compound according to claim 1.

11. A method for treating cancer in a subject in need thereof, comprising:
administering to the subject a therapeutically-effective amount of the compound according to claim 1.

12. The method according to claim 11, wherein the cancer comprises breast cancer.

13. A pharmaceutical composition of the GATA transcription factor proteolysis targeting chimera compound comprising the compound or pharmaceutically acceptable salts thereof according to claim 1, and a pharmaceutically acceptable carrier.

14. The pharmaceutical composition according to claim 13, wherein the pharmaceutical composition is preferably capsule, powder, tablet, granule, pill, injection, syrup, oral liquid, inhalant, ointment, suppository or patch.

15. A method for degrading a GATA transcription factor or treating GATA transcription factor abnormalities in a subject in need thereof, comprising:
administering to the subject a therapeutically-effective amount of the composition according to claim 13.

16. A method for degrading a GATA3 protein in a subject in need thereof, comprising:
administering to the subject a therapeutically-effective amount of the composition according to claim 13.

17. A method for treating cancer in a subject in need thereof, comprising:
administering to the subject a therapeutically-effective amount of the composition according to claim 13.

18. The method according to claim 17, wherein the cancer comprises breast cancer.
